(19) United States
(12) Patent Application Publication
(10) Pub. No.: US 2016/0160268 A1
(43) Pub. Date: Jun. 9, 2016

(54) AMDINOCILLIN FOR RAPID DETERMINATION OF SUSCEPTIBILITY TO BETA-LACTAM ANTIBIOTICS

(71) Applicants: THE REGENTS OF THE UNIVERSITY OF CALIFORNIA, Oakland, CA (US); UNITED STATES GOVERNMENT REPRESENTED BY THE DEPARTMENT OF VETERANS AFFAIRS, Washington, DC (US)

(72) Inventors: David A. HAAKE, Culver City, CA (US); Bernard M. CHURCHILL, PACIFI PALISADES, CA (US); Colin HALFORD, Los Angeles, CA (US)

(73) Assignees: THE REGENTS OF THE UNIVERSITY OF CALIFORNIA, Oakland, CA (US); UNITED STATES GOVERNMENT REPRESENTED BY THE DEPARTMENT OF VETERANS AFFAIRS, Washington, DC (US)

(21) Appl. No.: 14/907,182

(22) PCT Filed: Jul. 22, 2014
(86) PCT No.: PCT/US14/47684

§ 371 Date: Jan. 22, 2016

Related U.S. Application Data
(60) Provisional application No. 61/857,676, filed on Jul. 23, 2013.

(51) Int. Cl. C12Q 1/68

(52) U.S. Cl. CPC C12Q 1/689; C12Q 2600/136

(57) ABSTRACT

Described are methods for detecting susceptibility of a specimen to antibiotics, and particularly for enhancing such susceptibility testing for beta lactam antibiotics and antibiotics that bind to penicillin-binding proteins. The method comprises contacting the specimen with an oligonucleotide probe that specifically hybridizes with a target nucleic acid sequence region of ribosomal RNA. The target sequence is mature ribosomal RNA or at the splice site between a pre-ribosomal RNA tail and mature ribosomal RNA. Performing the method in the presence and absence of an antibiotic permits determination of antibiotic susceptibility. Rapid susceptibility testing is enabled by the addition of the PBP2-specific antibiotic, amdinocillin.

This application claims the benefit of U.S. provisional patent application No. 61/857,676, filed Jul. 23, 2013, the entire contents of which are incorporated herein by reference.

### STATEMENT REGARDING FEDERALLY SPONSORED RESEARCH

This invention was made with Government support under A1075565, awarded by the National Institutes of Health. The Government has certain rights in the invention. This work was supported by the U.S. Department of Veterans Affairs, and the Federal Government has certain rights in the invention.

### TECHNICAL FIELD OF THE INVENTION

The present invention relates generally to materials and methods for testing and determination of antibiotic susceptibility of bacteria in specimens of bodily fluid and other samples. The invention also relates to materials and methods for monitoring the physiological response of bacteria to antimicrobial agents.

### BACKGROUND

There is an urgent need for the development of rapid and convenient methods for detection and identification of antibiotic-resistant bacterial pathogens in clinical specimens to guide diagnosis and treatment of infectious diseases. Antibiotic therapy is based on identification of the pathogen and its antibiotic sensitivity. Treatment should not be delayed due to the seriousness of the disease, and thus is often started before this information is available. The effectiveness of individual antibiotics varies with the resistance of the bacterial pathogen to the antibiotic. Therapeutic outcomes can be significantly improved by the availability of a rapid assay for antibiotic susceptibility.

There remains an urgent need for the development of rapid and convenient methods for detection and testing of antibiotic susceptibility. In particular, there remains a need to develop methods for detecting susceptibility to beta-lactam antibiotics. The present invention addresses this need and others, as described below.

### SUMMARY

The invention provides a method for determining whether a sample of bacteria of interest is susceptible to an antibiotic agent, such as an antibiotic that preferentially binds to penicillin-binding proteins (PBPs). In one embodiment, the method comprises contacting a probe that specifically binds to a target sequence of ribosomal ribonucleic acid RNA (rRNA) or pre-ribosomal RNA (prRNA), of the bacteria of interest. In one embodiment, the target sequence comprises the junction, or splice site, between prRNA and mature ribosomal RNA (mrRNA). The probe can be a single probe or a pair of probes, such as a capture probe and a detector probe. In one embodiment, the probe is a single probe that specifically hybridizes to target sequence spanning the prRNA-mrRNA splice site. In another embodiment, the probe is a pair of probes that, collectively, specifically hybridize to a target sequence spanning the prRNA-mrRNA splice site. For example, one of the probes can hybridize to one side of the prRNA-mrRNA splice site while the other probe hybridizes to a contiguous length of target sequence of prRNA that spans the splice site. In some embodiments, the probe or probes hybridize to either the mrRNA or the prRNA. The probe is contacted with the sample both in the presence and in the absence of the antibiotic agent, and in the presence of a penicillin-binding protein (PBP) 2 specific-antibiotic, such as amdinocillin. A reduced amount of probe hybridization in the presence of the antibiotic agent relative to the amount of probe hybridization in the absence of the antibiotic agent is indicative of the susceptibility of the sample to antibiotic. On the other hand, a similar amount of probe hybridization in the presence of the antibiotic agent relative to the amount of probe hybridization in the absence of the antibiotic agent is indicative of resistance of the sample to the antibiotic. Alternatively, the amdinocillin can be administered to a patient concurrently with antibiotic treatment. The level of bacterial infection can then be monitored, and a reduction in the level of bacterial infection following treatment is indicative of infection caused by bacteria that are susceptible to antibiotic treatment.

In another embodiment, the method comprises contacting a specimen obtained from the sample of bacteria with an oligonucleotide probe or pair of probes in the absence of the agent. In one embodiment, the probe or pair of probes specifically hybridizes to a target sequence over the full length of the target sequence, wherein the target sequence consists of 25-35 contiguous nucleotides of bacterial ribosomal RNA (rRNA) spanning a splice site between a pre-ribosomal RNA (prRNA) tail and mature ribosomal RNA (mrRNA). The method further comprises contacting a specimen obtained from the sample with the probe or pair of probes in the presence of the antibiotic agent, and in the presence of a penicillin-binding protein (PBP) 2 specific-antibiotic, such as amdinocillin (also known as mecillinam); and detecting the relative amounts of probe hybridization to the target sequence in the specimens under the two contacting conditions. The sample is identified as susceptible to antibiotic treatment if the amount of probe hybridization to the target sequence in the presence of antibiotic is reduced by a significant amount relative to the amount of probe hybridization to the target sequence in the absence of antibiotic. Optionally, the method further comprises inoculating the specimen into a growth medium prior to the contacting steps.

The bacterial rRNA is 16S rRNA or 23S rRNA, or it can be 5S rRNA. Typically, the rRNA is 23SrRNA. The oligonucleotide probe or probes are typically each between about 10 to 50 nucleotides in length. In some embodiments, the probes are 12-30 nucleotides in length, while in other embodiments, they range in length from 14-20 nucleotides in length. Optionally, the oligonucleotide probe is labelled with a detectable marker. Representative markers include, but are not limited to, a fluorescent label, a radioactive label, a luminescent label, an enzyme, biotin, thiol or a dye. The detecting step of the method can comprise an optical, electrochemical or immunological assay, such as an enzyme-linked immunosorbent assay (ELISA).

In one embodiment, the method further comprises lysing the bacteria under conditions that release prRNA from the bacteria prior to the contacting steps. Thus, the sample can be prepared with a lysis agent present. Preferably, the lysis agent is selected so as to release prRNA but without damaging the target site. The targeting of the prRNA-mrRNA splice site means that the method can be performed without pre-treatment of the specimen to deplete prRNA prior to the contacting of probe with the sample, and without spliced prRNA tails interfering with the measurement. The ability to perform the method without such pre-treatment facilitates rapid processing of the susceptibility determination.

Antibiotic agents for susceptibility testing include, but are not limited to, Rifampicin, Chloramphenicol, aminoglycosides, quinolones, or beta-lactam antibiotics. In addition, novel or candidate antibiotic agents can be tested for efficacy using the methods described herein. In some embodiments, the method is used to guide diagnosis and treatment of a subject from whom the specimen containing bacteria has been obtained. For example, once the method has been employed to identify the antibiotic, or class of antibiotic, to which the specimen is susceptible, the method can further comprise administering the antibiotic to the subject.

Also provided is a method of enhancing and/or accelerating susceptibility testing for beta-lactam antibiotics and antibiotics that preferentially bind to penicillin-binding proteins (PBPs). The method comprises conducting a susceptibility test in the presence of a penicillin-binding protein (PBP) 2 specific-antibiotic, such as amdinocillin. This method is particularly suited for use with antibiotics that preferentially to PBPs, such as penicillin, cephalosporin, carbapenem, and monobactam antibiotics. Examples of antibiotics that preferentially bind to PBPs include, but are not limited to, ampicillin, piperacillin, ceftazidime, ceftriaxone, imipenem, and aztreonam.

A method for determining the antibiotic efficacy of a candidate antibiotic agent can comprise contacting a specimen obtained from the sample with an oligonucleotide probe or pair of probes in the absence of the agent, wherein the probe or pair of probes specifically hybridizes to a target sequence over the full length of the target sequence, wherein the target sequence comprises 25-35 contiguous nucleotides of either mature ribosomal RNA (mrRNA) or ribosomal RNA spanning a splice site between pre-ribosomal RNA (prRNA) tail and mrRNA, contacting a specimen obtained from the sample with the probe or pair of probes in the presence of the agent and in the presence of a PBP2 specific antibiotic, such as amdinocillin; and detecting the relative amounts of probe hybridization to the target sequence in the specimens. The agent is identified as effective if the amount of probe hybridization to the target sequence in the presence of the agent is reduced relative to the amount of probe hybridization to the target sequence in the absence of the agent. Typically, the reduction in the amount of probe hybridization is statistically significant, which in some cases will be an amount that is at least 10%, preferably at least 20%, and in some embodiments, the reduction is 30-90%.

The invention additionally provides a device for detecting mature rRNA or pre-rRNA in a bacterial sample. The device, in one embodiment, comprises an oligonucleotide probe immobilized on a solid support, wherein the oligonucleotide probe is between about 10 to 50 nucleotides in length and is capable of selectively hybridizing to a target sequence over the full length of the target sequence. The target sequence typically comprises 25-35 contiguous nucleotides of mature ribosomal RNA (mrRNA) or ribosomal RNA spanning a splice site between pre-ribosomal RNA (prRNA) tail and mrRNA. The solid support is typically an electrode or a membrane. Also contemplated is an ELISA well, or optical surface.

The invention further comprises a kit that can be used in practising the methods described herein. The kit can comprise an oligonucleotide probe or a pair of oligonucleotide probes selected from those described herein. The probes can optionally be labelled with a detectable marker. The kit can further comprise one or more containers for housing the probe(s) and other reagents for use with the method.

The invention also provides a method for monitoring the growth rate of a bacterial culture. The method comprises contacting a specimen obtained from the culture with a probe or pair of probes that specifically hybridizes to a target sequence over the full length of the target sequence, wherein the target sequence comprises 25-35 contiguous nucleotides of mature ribosomal RNA (mrRNA) or ribosomal RNA spanning a splice site between pre-ribosomal RNA (prRNA) tail and mrRNA. The method further comprises detecting the amount of probe hybridization to the target sequence in the specimen of (a) relative to an earlier time point; and/or relative to a control that either lacks or includes a growth medium component to be tested. The method can be performed in the presence of a PBP2-specific antibiotic, such as amdinocillin. The culture is identified as growing, or in a log phase of growth, if the amount of probe hybridization to the target sequence at the subsequent time point is increasing relative to the amount of probe hybridization to the target sequence at the earlier time point.

The invention additionally provides a method of determining whether a patient suffering from a bacterial infection has an infection caused by antibiotic-susceptible bacteria. The method comprises administering amdinocillin and a beta-lactam antibiotic to the patient; monitoring the level of bacterial infection in the patient; and determining that the patient suffers from an infection caused by antibiotic-susceptible bacteria if the level of bacterial infection is reduced following the administering of antibiotic and amdinocillin.

### BRIEF DESCRIPTION OF THE FIGURES

FIG. 1. Regions targeted by pre-rRNA probe pairs. The structures of the 16S (SEQ ID NO: 49) and 23S (SEQ ID NO: 50) pre-rRNA molecules are shown, including locations of mature rRNA termini (pointers) and regions targeted by electrochemical sensor probe pairs for the 16S 5′ tail (sequence between two heads of double-headed arrow) and 16S and 23S splice sites (between brackets).

FIG. 2. Comparison of pre-rRNA probe pairs. Probe pairs were tested for detection of E. coli in the log and stationary phases of growth. Log and stationary phase cells are expected to have high and low levels of pre-rRNA, respectively, yielding a high signal ratio for log vs. stationary phase cells. Probe pairs for 16S pre-rRNA had good sensitivity for log phase cells but the signal ratio for log vs. stationary phase cells was low. Some probe pairs targeting the splice sites at the termini of mature 16S and 23S rRNA had higher signal ratios for log vs. stationary phase cells. The probe pair targeting the splice site at the 3′ terminus of 23S rRNA had the best combination of sensitivity and high signal ratio of log vs. stationary phase cells.

FIGS. 3A-3B. Variations in pre-rRNA and rRNA levels during E. coli growth. FIG. 3A: Changes in mature rRNA, pre-rRNA, and their ratio were measured in overnight (ON) cultures that were subsequently inoculated into fresh MH growth medium and incubated for 7 hours at 37° C. FIG. 3B: Comparison of the mature/pre-RNA ratio and growth rate during different phases of growth. The growth rate curve is a weighted average determined from the change in CFU during each 30 min time period. Error bars estimated the standard deviation.

FIGS. 4A-4B. E. coli cell volume vs. rRNA copy number during different growth phases. FIG. 4A: Correlation between the cell volume and rRNA copy number per cell at densities below OD600 nm≦1.0. FIG. 4B: Electron micrographs demonstrating progressively smaller cells over incubation time from log phase (2.5 hrs) to stationary phase (7 hrs). Error bars estimated the standard deviation.

FIGS. 5A-5D. Response of mature rRNA and pre-rRNA to antibiotics. Antibiotics have differential effects on rRNA and pre-rRNA. Rifampicin (FIG. 5A) and ciprofloxacin (FIG. 5C) selectively inhibited transcription of new pre-rRNA, while addition of chloramphenicol (FIG. 5B) and gentamicin (FIG. 5D) resulted in a selective decrease in mature rRNA. Error bars estimated the standard deviation.

FIGS. 6A-6C. Comparison of ciprofloxacin susceptible and resistant E. coli strains. rRNA and pre-rRNA were measured in cultures of an E. coli clinical isolate susceptible to ciprofloxacin (EC103) and three ciprofloxacin resistant isolates (EC135, EC139, and EC197). The amount of pre-rRNA in strain EC103 was significantly lower than that of the ciprofloxacin resistant isolates within 15 min after addition of the antibiotic. Error bars estimated the standard deviation.

FIG. 7. Graph depicting the correlation between pre-rRNA copies per cell and bacterial growth rate. Growth rate is based on total cell volume as measured by turbidity or the increase in optical density at 600 nm, which peaks at 120 minutes, the same time as the peak in number of prRNA copies per cell.

FIG. 8. Evaluation of pre-rRNA probe pairs in gram-negative bacteria. The ratio of signals from probe pairs specific for pre-rRNA to mature rRNA were compared in overnight (O/N) or stationary phase cultures vs. cultures in the log phase of growth. Pre-rRNA signals were four-fold higher in log phase Klebsiella cells than in stationary phase Klebsiella cells, and six-fold higher in log phase Pseudomonas cells than in stationary phase Pseudomonas cells.

FIG. 9. Response of pre-rRNA to cefazolin. Addition of cefazolin to a culture of a susceptible strain of E. coli in the log phase of growth resulted in a one-log drop in the amount pre-rRNA within 30 min compared to a culture without the antibiotic. Error bars estimated the standard deviation.

FIG. 10. Flow chart of steps involved in detection of pre-rRNA.

FIGS. 11A-11C. Series of graphs depicting effects of ampicillin with or without amdinocillin on mature rRNA and pre-rRNA. Ampicillin-susceptible E. coli (strain EC135) was treated with ampicillin (16 μg/ml) alone, amdinocillin (1 μg/ml) alone, ampicillin plus amdinocillin, or neither. (11A) Effects on mature ribosomal RNA levels. (11B) Effects on pre-ribosomal RNA levels. (11C) Effects of ampicillin (16 μg/ml) plus amdinocillin (1 μg/ml) on pre-rRNA levels of ampicillin susceptible (EC135 and EC197) and resistant (EC96 and EC119) E. coli strains. Amdinocillin enabled early recognition of ampicillin susceptibility. Effects on pre-rRNA were more pronounced than those on mature rRNA.

FIGS. 12A-12B. Graphs illustrating effects of ceftriaxone with our without amdinocillin on mature rRNA and pre-rRNA. Ceftriaxone-susceptible E. coli (strain EC103) was treated with ceftriaxone (8 pg/ml) alone, amdinocillin (1 μg/ml) alone, ceftriaxone plus amdinocillin, or neither. (12A) Effects on mature ribosomal RNA levels. (12B) Effects on pre-ribosomal RNA levels. Ceftriaxone-mediated effects on mature rRNA and pre-rRNA occurred 60 min faster with amdinocillin than without amdinocillin.

FIGS. 13A-13C. Graphs showing that effects on pre-rRNA are concentration dependent. Cefazolin-susceptible E. coli (strain EC103) was treated with amdinocillin (1 μg/ml) plus cefazolin at concentrations ranging from 0-32 μg/ml. (13A) At a cefazolin concentration of 32 μg/ml, amdinocillin had no effect on pre-RNA levels. (13B) At a cefazolin concentration of 4 μg/ml, amdinocillin enabled early recognition of cefazolin-susceptibility. (13C) Pre-rRNA levels fell more quickly at higher cefazolin concentrations.

FIGS. 14A-14D. Graphs illustrating the effects of beta-lactam antibiotics plus amdinocillin on antibiotic susceptible and resistant bacteria. Antibiotic susceptible and resistant bacteria were treated with amdinocillin (1 μg/ml) plus various beta lactam antibiotics including 16 pg/ml cefazolin (14A), 4 μg/ml ceftriaxone (14B), 32 μg/ml piperacillin plus 4 pg/ml tazobactam (14C), and 2 μg/ml imipenem (14D). Differential effects on pre-rRNA of susceptible from resistant bacteria were evident within 30-90 min.

FIGS. 15A-15F. Digital photomicrographs showing results of Kirby-Bauer disc diffusion antibiotic-susceptibility tests. 6 mm diameter antibiotic discs were placed on agar plates seeded with a lawn of E. coli that were (15A) Ampicillin susceptible (strain EC135), (15B) Ampicillin resistant (strain EC96), (15C) Cefazolin susceptible (strain EC103), (15D) Cefazolin resistant (strain EC96), (15E) Imipenem susceptible (strain EC103) or (15F) Imipenem resistant (strain NDM-1). In each panel the antibiotic disc is on the left and the amdinocillin disc is on the right. Images were obtained after 20 hours of incubation at 37° C. Synergy was not observed between amdinocillin and any of the antibiotics tested.

FIGS. 16A-16D. Demonstration that amdinocillin blocks ampicillin-induced E. coli filamentation. The lengths of ampicillin-susceptible E. coli (strain EC103) cells were measured after treatment for 30 minutes with no antibiotics, ampicillin, or ampicillin plus amdinocillin. Digital photomicrographs of representative cells treated with no antibiotics (16A), ampicillin (16B), and ampicillin plus amdinocillin (16C) are shown. FIG. 16D is a frequency histogram showing that ampicillin caused an average 4-fold increase in length of E. coli cells, which was partially blocked by amdinocillin.

### DETAILED DESCRIPTION

Ribosomal RNA is an excellent target molecule for pathogen detection systems because of its abundance in the bacterial cell and because of the accessibility of species-specific signature sequences to probe hybridization (6). (Numbers in parentheses correspond to numbers in list of cited references at the end of the Detailed Description.) When combined with sensitive surface chemistry methods to minimize nonspecific background signals, such rRNA probe hybridization sensors are able to detect as few as 100 bacteria per ml (2, 7, 16). Estimations of bacterial density are possible because, within the dynamic range of the assay, there is a log-log correlation between the concentration of target rRNA molecules in the bacterial lysate and the amperometric current amplitude generated by the electrochemical sensor assay (9, 11). The accuracy of bacterial quantitation methods based on rRNA detection is mitigated by variations in the number of rRNA molecules per cell depending on the cell type and bacterial growth phase. In E. coli, the rRNA copy number per cell has been estimated to vary from as high as 72,000 during log phase to less than 6,800 during stationary phase (1).

Precursor ribosomal RNA (pre-rRNA) is an intermediate stage in the formation of mature ribosomal RNA (rRNA) and is a useful marker for cellular metabolism and growth rate. In one embodiment, the invention provides an electrochemical sensor assay for Escherichia coli pre-rRNA involving hybridization of capture and detector probes with tail sections that are spliced away during rRNA maturation. A ternary self-assembled monolayer (SAM) prepared on gold electrodes surfaces by co-assembling of thiolated capture probes with hexanedithiol and post-treatment with 6-mercapto-1-hexanol minimized background signal and maximized the signal-to-noise ratio. Inclusion of internal calibration controls allowed accurate estimation of the pre-rRNA copy number per cell. As expected, the ratio of pre-rRNA to mature rRNA was low during stationary phase and high during log phase. Pre-rRNA levels were highly dynamic, ranging from 2 copies per cell during stationary phase to ˜1200 copies per cell within 60 min of inoculation into fresh growth medium. Specificity of the assay for pre-rRNA was validated using rifampicin and chloramphenicol, which are known inhibitors of pre-rRNA synthesis and processing, respectively. The DNA gyrase inhibitor, ciprofloxacin, was found to act similarly to rifampicin; a decline in pre-rRNA was detectable within 15 minutes in ciprofloxacin susceptible bacteria. The invention provides assays for pre-rRNA, which provide insights into cellular metabolism as well as predictors of antibiotic susceptibility.

To address the need for antibiotic resistance data at the time of initial antibiotic selection, methods are described herein to analyze the antibiotic susceptibility of organisms in clinical specimens. The invention thus provides a method for detecting and identifying antibiotic susceptibility in a specimen containing, or suspected of containing, bacteria. In some embodiments, the method is used to guide diagnosis and treatment of a subject from whom the specimen containing bacteria has been obtained. For example, once the method has been employed to identify the antibiotic, or class of antibiotic, to which the specimen is susceptible, the method can further comprise administering the antibiotic to the subject.

### Definitions

All scientific and technical terms used in this application have meanings commonly used in the art unless otherwise specified. As used in this application, the following words or phrases have the meanings specified.

As used herein, an “oligonucleotide probe” is an oligonucleotide having a nucleotide sequence sufficiently complementary to its target nucleic acid sequence to be able to form a detectable hybrid probe:target duplex under high stringency hybridization conditions. An oligonucleotide probe is an isolated chemical species and may include additional nucleotides outside of the targeted region as long as such nucleotides do not prevent hybridization under high stringency hybridization conditions. Non-complementary sequences, such as promoter sequences, restriction endonuclease recognition sites, or sequences that confer a desired secondary or tertiary structure such as a catalytic active site can be used to facilitate detection using the invented probes. An oligonucleotide probe optionally may be labeled with a detectable marker such as a radioisotope, a fluorescent moiety, a chemiluminescent moiety, an enzyme or a ligand, which can be used to detect or confirm probe hybridization to its target sequence. “Probe specificity” refers to the ability of a probe to distinguish between target and non-target sequences.

The term “nucleic acid”, “oligonucleotide” or “polynucleotide” refers to a deoxyribo-nucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, a “detectable marker” or “label” is a molecule attached to, or synthesized as part of a nucleic acid probe. This molecule should be uniquely detectable and will allow the probe to be detected as a result. These detectable moieties are often radioisotopes, chemiluminescent molecules, enzymes, haptens, or even unique oligonucleotide sequences.

As used herein, a “hybrid” or a “duplex” is a complex formed between two single-stranded nucleic acid sequences by Watson-Crick base pairings or non-canonical base pairings between the complementary bases.

As used herein, “hybridization” is the process by which two complementary strands of nucleic acid combine to form a double-stranded structure (“hybrid” or “duplex”). “Stringency” is used to describe the temperature and solvent composition existing during hybridization and the subsequent processing steps. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. Stringency conditions are chosen to maximize the difference in stability between the hybrid formed with the target and the non-target nucleic acid. Exemplary stringency conditions are described herein below.

As used herein, “complementarity” is a property conferred by the base sequence of a single strand of DNA or RNA which may form a hybrid or double-stranded DNA:DNA, RNA:RNA or DNA:RNA through hydrogen bonding between Watson-Crick base pairs on the respective strands. Adenine (A) ordinarily complements thymine (T) or Uracil (U), while guanine (G) ordinarily complements cytosine (C). “Fully complementary”, when describing a probe with respect to its target sequence, means that complementarity is present along the full length of the probe.

As used herein, “adjacent”, in the context of nucleotide sequences and oligonucleotides, means immediately next to one another (end to end), such that two adjacent molecules do not overlap with one another and there is no gap between them. For example, two oligonucleotide probes hybridized to adjacent regions of a target nucleic acid molecule have no nucleotides of the target sequence (unpaired with either of the two probes) between them.

As used herein, the phrases “consist essentially of” or “consisting essentially of” mean that the oligonucleotide has a nucleotide sequence substantially similar to a specified nucleotide sequence. Any additions or deletions are non-material variations of the specified nucleotide sequence which do not prevent the oligonucleotide from having its claimed property, such as being able to preferentially hybridize under high stringency hybridization conditions to its target nucleic acid over non-target nucleic acids.

One skilled in the art will understand that substantially corresponding probes of the invention can vary from the referred-to sequence and still hybridize to the same target nucleic acid sequence. This variation from the nucleic acid may be stated in terms of a percentage of identical bases within the sequence or the percentage of perfectly complementary bases between the probe and its target sequence. Probes of the present invention substantially correspond to a nucleic acid sequence if these percentages are from 100% to 80% or from 0 base mismatches in a 10 nucleotide target sequence to 2 bases mismatched in a 10 nucleotide target sequence. In preferred embodiments, the percentage is from 100% to 85%. In more preferred embodiments, this percentage is from 90% to 100%; in other preferred embodiments, this percentage is from 95% to 100%.

By “sufficiently complementary” or “substantially complementary” is meant nucleic acids having a sufficient amount of contiguous complementary nucleotides to form, under high stringency hybridization conditions, a hybrid that is stable for detection.

By “preferentially hybridize” is meant that, under high stringency hybridization conditions, oligonucleotide probes can hybridize with their target nucleic acids to form stable probe:target hybrids (thereby indicating the presence of the target nucleic acids) without forming stable probe:non-target hybrids (that would indicate the presence of non-target nucleic acids from other organisms). Thus, the probe hybridizes to target nucleic acid to a sufficiently greater extent than to non-target nucleic acid to enable one skilled in the art to accurately detect the presence of the relevant bacteria and distinguish their presence from that of other organisms. Preferential hybridization can be measured using techniques known in the art and described herein.

As used herein, a “target nucleic acid sequence region” of a pathogen refers to a nucleic acid sequence present in the nucleic acid of an organism or a sequence complementary thereto, which is not present in the nucleic acids of other species. Nucleic acids having nucleotide sequences complementary to a target sequence may be generated by target amplification techniques such as polymerase chain reaction (PCR) or transcription mediated amplification.

As used herein, “room temperature” means about 20-25° C.

As used herein, “a” or “an” means at least one, unless clearly indicated otherwise.

### Probes of the Invention

The invention provides oligonucleotide probes that are specific for bacterial rRNA. In a typical embodiment, the probes are fully complementary to the target sequence.

Representative target sequences for probe hybridization with rRNA of indicated bacterial species are presented below:

E. coli (all enterobacteriaceae) target sequence:

(SEQ ID NO: 1)

AATGAACCGTGAGGCTT|AACCTTACAACGCCGAAGCTGTTTTGGCGG
ATTG;

Pseudomonas aeruginosa target sequence:

(SEQ ID NO: 2)

AATTGCCCGTGAGGCTT|GACCATATAACACCCAAACAATCTGACGA
TTGT;

Streptococcus pyogenes target sequence:

(SEQ ID NO: 3)
AATAGCTCGAGGACTT|ATCCAAAAAGAAATATTGACAACGTTACGGA
TTCTTG;

Staphylococcus aureus target sequence:

(SEQ ID NO: 4)
AATCGATCGAAGACTT|AATCAAAATAAATGTTTTGCGAAGCAAAATCA
CTT;

wherein I indicates the splice site between prRNA and mRNA.

Representative probe pairs directed to these target sequences include the following:

E. coli (all enterobacteriaceae) probes

(SEQ ID NO: 5)

5′-AAGCCTCACGGTTCATT
and

(SEQ ID NO: 6)

GGCGTTGTAAGGTT;

Pseudomonas aeruginosa probes:

(SEQ ID NO: 7)

5′-AAGCCTCACGGGCAATT
and

(SEQ ID NO: 8)

GGTGTTATATGGTC;

Streptococcus pyogenes probes:

(SEQ ID NO: 9)

AAGTCCTCGAGCTATT
and

(SEQ ID NO: 10)

ATTTCTTTTTGGAT;
and

Staphylococcus aureus probes

(SEQ ID NO: 11)

AAGTCTTCGATCGATT
and

(SEQ ID NO: 12)

CATTTATTTTGATT.

Oligonucleotides may be prepared using any of a variety of techniques known in the art. Oligonucleotide probes of the invention include the sequences shown above and in Table 1 of the Example below, and equivalent sequences that exhibit essentially the same ability to form a detectable hybrid probe:target duplex under high stringency hybridization conditions. Oligonucleotide probes typically range in size from 10 to 50 nucleotides in length. Preferred probes are 10-35 nucleotides in length, with 10-25 nucleotides being optimal for some conditions. For example, hybridization at room temperature allows for use of shorter probes, typically with 5-6 nucleotides on either side of the splice site between pre-rRNA and mature rRNA. Hybridization at higher temperatures, such as 50° C., would call for longer probes, typically with 10 or more nucleotides on either side of the splice site. A variety of detectable labels are known in the art, including but not limited to, enzymatic, fluorescent, and radioisotope labels.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

An advantage of the probes of the invention is their ability to hybridize to the target sequence with sufficient selectivity and strength at ambient temperature and without requiring the use of a denaturing agent. The probes of the invention can be used to detect species-specific targets at room temperature (or at body temperature), at native pH (7.0) in a 1 M phosphate buffer. Accordingly, for the short (10-35 bases in length) probes of the invention, “highly stringent conditions” include hybridization and washes at 20° C. to 39° C. in 1 M phosphate buffer, or other buffer containing an appropriate salt solution, at native pH (at or near 7.0).

Suitable “moderately stringent conditions” include prewashing in a solution of 5 X SSC, 0.5% SDS, 1.0 mM EDTA (pH 8.0); hybridizing at 50° C.-65° C., 5×SSC, overnight; followed by washing twice at 65° C. for 20 minutes with each of 2X, 0.5X and 0.2X SSC containing 0.1% SDS.

Any polynucleotide may be further modified to increase stability. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences can be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include probe generation vectors. In general, a vector will contain an origin of replication functional in at least one organism, convenient restriction endonuclease sites and one or more selectable markers. Other elements will depend upon the desired use, and will be apparent to those of ordinary skill in the art.

### Methods of Detecting Antibiotic Susceptibility

The invention provides a method for determining whether a sample of bacteria of interest is susceptible to an antibiotic agent. In one embodiment, the method comprises contacting a probe that specifically binds to a target sequence of ribosomal RNA (rRNA) or pre-ribosomal ribonucleic acid (prRNA), of the bacteria of interest. In one embodiment, the target sequence comprises the junction, or splice site, between prRNA and mature ribosomal RNA (mrRNA). The probe can be a single probe or a pair of probes, such as a capture probe and a detector probe. In one embodiment, the probe is a single probe that specifically hybridizes to a target sequence spanning the prRNA-mrRNA splice site. In another embodiment, the probe is a pair of probes that, collectively, specifically hybridize to a target sequence spanning the prRNA-mrRNA splice site. For example, one of the probes can hybridize to either side of the prRNA-mrRNA splice site while the other probe hybridizes to a contiguous length of target sequence of prRNA that spans the splice site. The probe is contacted with the sample both in the presence and in the absence of the antibiotic agent. A reduced amount of probe hybridization in the presence of the antibiotic agent relative to the amount of probe hybridization in the absence of the antibiotic agent is indicative of the susceptibility of the sample to antibiotic.

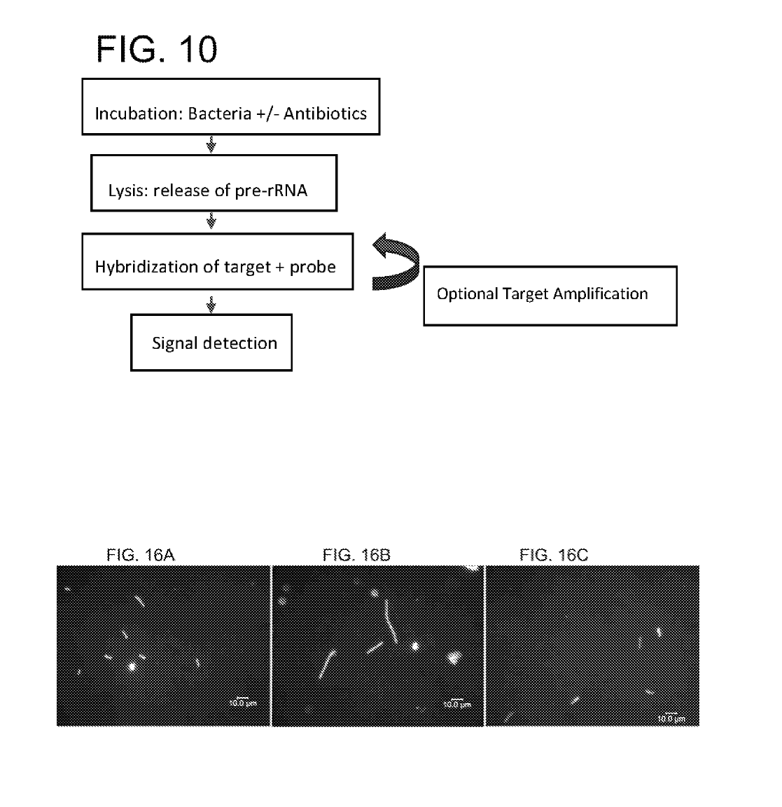

The steps of the method are summarized in the flow diagram shown in FIG. 10. First, the bacteria are incubated in the presence of the antibiotics to be tested. A lysis step releases the mature rRNA and prRNA from the bacteria. Various methods of lysis are known in the art and include, but are not limited to, alkaline lysis, enzymatic lysis, sonication, homogenization, and electrolysis. Rapid lysis and methods of lysis that optimize preservation of RNA are preferred. Probes are then brought into contact with the prRNA for hybridization with target sequence. As is understood to those skilled in the art, the use of a single probe or a pair of probes will be influenced by the signal detection method in use. For example, a single probe may be selected for use with a luminescent signal, while electrochemical detection benefits from the use of a pair of probes (e.g., capture and detection probes). Optionally, the target is amplified, e.g., using PCR. It is possible, however, to detect, for example, 250 bacteria/ml without use of PCR. Detection of signal associated with the target sequence via probe hybridization can be accomplished through various means known in the art.

This method described above, as well as other methods described herein, can be enhanced by performing the contacting and/or hybridization in the presence of a penicillin-binding protein (PBP) 2 specific antibiotic, such as amdinocillin. This enhancement is beneficial for assays involving antibiotics, such as beta-lactam antibiotics, whose efficacy can be delayed if the antibiotic is effective against division but not elongation of the target pathogen. The enhancement created by adding the amdinocillin to the incubation can be effective with probes directed to the mature RNA as well as with probes directed to the pre-rRNA (spanning the splice site). The method can be performed using a specimen obtained from a patient being treated for bacterial infection with antibiotics. The method can be used to determine whether the bacteria causing the patient's infection is susceptible to antibiotic therapy. Alternatively, amdinocillin can be administered to a patient concurrently with antibiotic treatment. The level of bacterial infection can then be monitored, and a reduction in the level of bacterial infection following treatment is indicative of infection caused by bacteria that are susceptible to antibiotic treatment.

In another embodiment, the method comprises contacting a specimen obtained from the sample of bacteria with an oligonucleotide probe or pair of probes in the absence of the agent. In one embodiment, the probe or pair of probes specifically hybridizes to a target sequence over the full length of the target sequence, wherein the target sequence consists of 25-35 contiguous nucleotides of bacterial ribosomal RNA (rRNA) spanning a splice site between a pre-ribosomal RNA (prRNA) tail and mature ribosomal RNA (mrRNA). The method further comprises contacting a specimen obtained from the sample with the probe or pair of probes in the presence of the antibiotic agent; and detecting the relative amounts of probe hybridization to the target sequence in the specimens under the two contacting conditions. Optionally, the method further comprises inoculating the specimen into a growth medium prior to the contacting steps.

The sample is identified as susceptible to antibiotic treatment if the amount of probe hybridization to the target sequence in the presence of antibiotic is reduced sufficiently to meet the standard set forth by the U.S. Food and Drug Administration for Antimicrobial Susceptibility Test (AST) Systems. For example, >90% essential and category agreement, <3% major errors, and <1.5% very major errors, when compared to standard clinical microbiology methods. The amount of reduced probe hybridization that meets this criterion will vary with the test conditions. In one embodiment, the amount of reduction will be at least 10% or at least 20% relative to the amount of probe hybridization to the target sequence in the absence of antibiotic. In another embodiment, the amount of hybridization to the target sequence is reduced by 30%, 40%, 50%, 60%, 70%, 80%, 90%, or 95%, relative to the amount of probe hybridization to target sequence in the absence of antibiotic.

The bacterial rRNA is 16S rRNA or 23S rRNA, or it can be 5S rRNA. Typically, the rRNA is 23S rRNA. The oligonucleotide probe or probes are typically each between about 10 to 50 nucleotides in length. In some embodiments, the probes are 12-30 nucleotides in length, while in others they range in length from 14-20 nucleotides in length. Optionally, the oligonucleotide probe is labelled with a detectable marker. Representative markers include, but are not limited to, a fluorescent label, a radioactive label, a luminescent label, an enzyme, biotin, thiol or a dye. The detecting step of the method can comprise an optical, electrochemical or immunological assay.

In one embodiment, the method further comprises lysing the bacteria under conditions that release rRNA from the bacteria prior to the contacting steps. Thus, the sample can be prepared with a lysis agent present. Preferably, the lysis agent is selected so as to release rRNA but without damaging the rRNA. The targeting of the prRNA-mrRNA splice site means that the method can be performed without pre-treatment of the specimen to deplete prRNA prior to the contacting of probe with the sample, and without spliced prRNA tails interfering with the measurement. The ability to perform the method without such pre-treatment facilitates rapid processing of the susceptibility determination.

Antibiotic agents for susceptibility testing include, but are not limited to, Rifampicin, Chloramphenicol, aminoglycosides, quinolones, or beta-lactam antibiotics. In addition, novel or candidate antibiotic agents can be tested for efficacy using the methods described herein. The invention additionally provides a method of treating a subject having, or suspected of having, a bacterial infection. The method comprises determining the antibiotic susceptibility in a specimen obtained from the subject as described herein, and administering to the subject an antibiotic to which the specimen is susceptible.

A method for determining the antibiotic efficacy of a candidate antibiotic agent can comprise contacting a specimen obtained from the sample with an oligonucleotide probe or pair of probes in the absence of the agent, wherein the probe or pair of probes specifically hybridizes to a target sequence over the full length of the target sequence, wherein the target sequence comprises 25-35 contiguous nucleotides of bacterial ribosomal RNA (rRNA) spanning a splice site between pre-ribosomal RNA (prRNA) tail and mature ribosomal RNA (mrRNA), contacting a specimen obtained from the sample with the probe or pair of probes in the presence of the agent; and detecting the relative amounts of probe hybridization to the target sequence in the specimens. The agent is identified as effective if the amount of probe hybridization to the target sequence in the presence of the agent is reduced by at least 20% relative to the amount of probe hybridization to the target sequence in the absence of the agent.

Bacteria contained within the specimen can be lysed using one of the lysis preparations described herein. In one embodiment, the lysis preparation comprises a universal lysis method consisting of two steps: a first step involving treatment of bacteria with a buffer containing 1% Triton X-100, 0.1 M KH₂PO₄, 2 mM EDTA and 1 mg/ml lysozyme, followed by a second step involving treatment with 1 M NaOH. Use of the universal lysis method obviates the need to use separate lysis buffer for gram-positive and gram-negative bacteria. In this embodiment, the time-consuming steps of bacterial RNA and/or DNA purification are not necessary, permitting direct application of a lysed urine sample to the capture probes, improving speed and efficiency of the assay. Accordingly, the method can be performed by first lysing a specimen of interest to release nucleic acid molecules of the pathogen.

Alternatively, the lysate can be prepared by contacting the specimen with either a first lysis buffer comprising a non-denaturing detergent (e.g., Triton X-100) and lysozyme, or a second lysis buffer comprising NaOH. Typically, the Triton X-100 is used at 0.1%, lysozyme at 1 mg/ml, and NaOH at 1 M. In another embodiment, the lysing comprises contacting the specimen with both buffers in series, e.g. with the second lysis buffer, either before or after contacting the specimen with the first lysis buffer. The contacting of the specimen with the buffer(s) typically occurs at room temperature. Typically, the specimen is in contact with the lysis buffer for a total of about 10 minutes. Where a first and second lysis buffer is used, the contact with each buffer is typically about 5 minutes. Those skilled in the art are aware that the time and temperature under which the contact with lysis buffer occurs can be varied (e.g. higher temperatures will accelerate the lysis) and also optimized for a particular specimen, target pathogen and other assay conditions.

The method comprises contacting a specimen with one or more detector probes of the invention under conditions permitting hybridization of target nucleic acid molecules of pathogens (e.g., bacteria) present in the specimen with the detector probes, resulting in hybridized target nucleic acid molecules. One or more hybridized target probes are brought into contact with one or more capture probes, under conditions permitting hybridization of capture probes with target nucleic acid molecules.

Accordingly, the target nucleic acid ultimately hybridizes with both capture probe(s) and detector probe(s). Although these two hybridization steps can be performed in any order, in one embodiment, detector probe hybridizes with the target nucleic acid first, after which the hybridized material is brought into contact with an immobilized capture probe. Following a wash, the dectector:target:capture combination is immobilized on a surface to which the capture probe has been bound. Detection of probe bound to target nucleic acid is indicative of presence of pathogen.

For use with an electrochemical sensor, such as the sensor array available from GeneFluidics, Inc. (Monterey Park, Calif.), the method comprises detection of current associated with binding of probe to target. In one embodiment illustrated in the example below, the capture probe is labeled with biotin and immobilized onto a surface treated with streptavidin. The detector probe in this example is tagged with fluorescein, providing an antigen to which a horse radish peroxidase-labeled antibody binds. This peroxidase, in the presence of its substrate (typically, hydrogen peroxide and tetramethylbenzidine), catalyzes a well-characterized redox reaction and generates a measurable electroreduction current under a fixed voltage potential, thereby providing an electrochemical signal to detect presence of the target nucleic acid. Those skilled in the art are aware of alternative labels and enzymes that can be used in an electrochemical assay.

Preferably, the method for detecting antibiotic resistance is performed after first identifying and quantifying the pathogen of interest. The method of detecting the presence of a pathogen set forth in U.S. Pat. No. 7,763,426 can be used to identify the pathogen. Identification of the pathogen guides the selection of antibiotic to be tested for resistance.

Quantitation of the pathogen guides the selection of an appropriate ratio of antibiotic to pathogen for subsequent testing. The method is then carried out by inoculation of the pathogen-containing specimen into a growth medium. The inoculation is performed at a dilution determined by the results of the quantitation. This inoculation is preferably done in both the presence and absence of antibiotic. The presence or amount of pathogen is then determined, typically by comparing the specimens inoculated in the presence and in the absence of antibiotic. A greater pathogen amount in the presence of antibiotic is indicative of resistance to the antibiotic. The comparison is typically based on comparing the amount of labeled oligonucleotide (detector probe) complexed with the substrate for inoculations into growth medium in the presence and absence of antibiotic.

### Methods of Monitoring Bacterial Growth Rate

The invention also provides a method for monitoring the growth rate of a bacterial culture. The method comprises contacting a specimen obtained from the culture with a probe or pair of probes that specifically hybridizes to a target sequence over the full length of the target sequence, wherein the target sequence comprises 25-35 contiguous nucleotides of bacterial ribosomal RNA (rRNA) spanning a splice site between pre-ribosomal RNA (prRNA) tail and mature ribosomal RNA (mrRNA). The method further comprises detecting the amount of probe hybridization to the target sequence in the specimen relative to an earlier time point; and/or relative to a control that either lacks or includes a growth medium component to be tested. The culture is identified as growing, or in a log phase of growth, if the amount of probe hybridization to the target sequence at the subsequent time point is increasing relative to the amount of probe hybridization to the target sequence at the earlier time point.

### Kits and Devices

The invention additionally provides a device for detecting mature rRNA or pre-rRNA in a bacterial sample. The device, in one embodiment, comprises an oligonucleotide probe immobilized on a solid support, wherein the oligonucleotide probe is between about 10 to 50 nucleotides in length and is capable of selectively hybridizing to a target sequence over the full length of the target sequence. The target sequence typically comprises 25-35 contiguous nucleotides of mature ribosomal RNA (mrRNA) or ribosomal RNA spanning a splice site between pre-ribosomal RNA (prRNA) tail and mrRNA. The solid support is typically an electrode or a membrane. Also contemplated is an ELISA well, or optical surface.

The invention further comprises a kit that can be used in practising the methods described herein. The kit can comprise an oligonucleotide probe or a pair of oligonucleotide probes selected from those described herein. The probes can optionally be labelled with a detectable marker. The kit can further comprise one or more containers for housing the probe(s) and other reagents for use with the method. The invention additionally provides an assay kit for use in carrying out the method of the invention. The kit comprises one or more of the probes described herein, and, optionally, a container or substrate. In one embodiment, the kit comprises a substrate to which one or more capture probes of the invention are bound or otherwise immobilized. Optionally, the kit further comprises a container and one or more detector probes corresponding to the capture probes. In one embodiment, the substrate is an electrochemical sensor array.

### EXAMPLES

The following examples are presented to illustrate the present invention and to assist one of ordinary skill in making and using the same. The examples are not intended in any way to otherwise limit the scope of the invention.

### Example 1

Rapid Antimicrobial Susceptibility Testing by Sensitive Detection of Precursor rRNA Using an Electrochemical Biosensing Platform

Ribosomal RNA is an excellent target molecule for pathogen detection systems because of its abundance in the bacterial cell and because of the accessibility of species-specific signature sequences to probe hybridization (6). When combined with sensitive surface chemistry methods to minimize nonspecific background signals, such rRNA probe hybridization sensors are able to detect as few as 100 bacteria per ml (2, 8, 17). Estimations of bacterial density are possible because, within the dynamic range of the assay, there is a log-log correlation between the concentration of target rRNA molecules in the bacterial lysate and the amperometric current amplitude generated by the electrochemical sensor assay (10, 12). The accuracy of bacterial quantitation methods based on rRNA detection is mitigated by variations in the number of rRNA molecules per cell depending on the cell type and bacterial growth phase. In E. coli, the rRNA copy number per cell has been estimated to vary from as high as 72,000 during log phase to less than 6,800 during stationary phase (1).

Electrochemical sensors have the potential to rapidly determine antibiotic susceptibility by monitoring the phenotypic response of bacteria to antibiotics. Cellular pre-rRNA levels would be expected to fall as antibiotics shift the cellular metabolism of antibiotic-susceptible bacteria from log phase to stationary phase. The size of the pre-rRNA pool in the cell is determined by the synthesis and degradation rates, which are directly or indirectly affected by antibiotics (3). For this reason, we developed and validated an electrochemical assay for pre-rRNA determination. By calibrating sensor signal intensities with an internal standard, and correlating these signals with bacterial density, we were able to estimate the number of rRNA and pre-rRNA copies per cell. Our studies provide new insight into the kinetics of rRNA and pre-rRNA levels during bacterial growth phases, and in response to certain antibiotics. Of interest, we determined that pre-rRNA and/or rRNA levels rapidly respond to the quinolone antibiotic, ciprofloxacin, and the aminoglycoside antibiotic, gentamicin, in susceptible E. coli.

### Materials & Methods

### Bacterial Strains and Media.

E. coli clinical urine isolate EC103 (Amp^{R}) was obtained from the University of California-Los Angeles (UCLA) Clinical Microbiology Laboratory with approval from the UCLA and Veterans' Affair Institutional Review Boards and appropriate Health Insurance Portability and Accountability Act exemptions. EC103 was inoculated into Mueller Hinton (MH) broth with 12% glycerol (Becton Dickinson, Sparks, Md.) and stored at −80° C. EC103 was cultured overnight in MH broth with 64 μg/ml ampicillin (Sigma, St. Louis, Mo.). EC103 was plated on Luria Broth (LB) agar (MOBIO Laboratories Inc., Carlsbad, Calif.) for counting colony-forming units (CFUs).

### EC103 Growth and Target Copy Number Experiments.

Overnight cultures of EC103 were prepared by adding 5 μl of EC103 glycerol stock to 5 ml of MH broth with ampicillin and incubated at 37° C. overnight with shaking. The following day, the EC103 culture was diluted by adding 10 μl of the overnight culture to 100 ml of prewarmed and preshaken MH broth in a 500 ml flask, followed by incubation at 37° C. with shaking at 250 rpm. Every 30 min, including at 0 min and the overnight culture itself, a 1 ml sample was taken for OD₆₀₀ measurement and 10-fold serial dilutions (100 μl into 900 μl) were performed in room temperature MH broth. Cell density was determined by plating serial dilutions in triplicate. At each time point, culture samples were transferred to an ice water bath or centrifuged immediately at 4° C. for 3 min at 14,000 rpm. The supernatants were then removed by aspiration, flash frozen in a dry ice-ethanol bath and stored at −80° C.

In certain growth experiments, one culture was spiked with one of the following antibiotics at either 150 or 210 minutes: 25 μg/ml Rifampicin (Sigma, St. Louis, Mo.), 25 μg/ml Chloramphenicol (Sigma, St. Louis, Mo.), 4 μg/ml Ciprofloxacin (Sigma, St. Louis, Mo.) or 16 μg/ml Gentamicin (Sigma, St. Louis, Mo.). After addition of antibiotics at 150 minutes, samples were collected every 15 min instead of every 30 min.

For experiments comparing pre-ribosomal probe sensitivity and specificity, culture samples were taken from the overnight culture and the EC103 culture at log phase (OD₆₀₀=0.1) and centrifuged immediately at 4° C. for 5 min at 14,000 rpm. Supernatants were removed by aspiration. The pellets were flash frozen in a dry ice-ethanol bath and stored at −80° C.

Electrochemical detection. Electrochemical detection of bacterial rRNA and pre-rRNA was performed as previously described for biotinylated (12) and thiolated capture probes (2, 8) immobilized on photolithographically prepared Au electrode arrays, with modifications.

The sensor response was evaluated with a sandwich-type hybridization assay, using fluorescein (FITC) as a tracer in the detection probe and anti-FITC-horseradish peroxidase (HRP) as the reporter molecule. 3,3′5,5′-tetramethylbenzidine (TMB)-H₂O₂ was the selected substrate for the electrochemical measurement of the activity of the captured HRP reporter. All synthetic oligonucleotides used were purchased from Eurofins MWG Operon and are listed in Table 1. For thiolated capture probes, disposable 16-sensor bare Au electrode arrays were obtained from GeneFluidics (Irwindale, Calif.). Each sensor of the array consisted of a 2.5 mm diameter central working electrode, surrounded by an Au counter electrode and an Au pseudo-reference electrode. The sensor chip was driven by a computer-controlled Helios multichannel electrochemical workstation (GeneFluidics, Irwindale, Calif.). Washing steps were carried out after each application of reagents by applying a stream of deionized H₂O to the sensor surface for approximately 2-3 sec followed by 5 sec of drying under a stream of nitrogen. Prior to addition of the first reagent, the bare gold chips were dried as described above. To functionalize the working sensor surface, a fresh mixture of 0.05 μM thiolated capture probe and 300 μM 1,6-hexanedithiol (96%, Sigma, St. Louis, Mo.) was prepared in 10 mM Tris-HCl, 1 mM EDTA and 0.3 M NaCl (pH 8.0) and allowed to stand at room temperature for 10 min. Aliquots of 6 μl of this mixture were cast over each Au working electrode in the 16-sensor array and incubated overnight at 4° C. in a humidified chamber.

Unless otherwise stated, all subsequent steps were performed at room temperature. The following day, the mixed monolayer-modified Au sensors were subsequently treated with 6 pl of 1 mM 6-Mercapto-1-hexanol (97%, Sigma, St. Louis, Mo.) in 10 mM Tris-HCl, 1 mM EDTA and 0.3 M NaCl (pH 8.0) for 50 min to obtain the ternary monolayer interface.

For biotinylated capture probes, 16-sensor Au electrode arrays precapped with a binary SAM consisting of mercaptohexanol and mercaptoundecanoic acid in a 5:1 ratio were obtained from GeneFluidics (Irwindale, Calif.). Washing steps were carried out after each application of reagents by applying a stream of deionized H₂O to the sensor surface for approximately 2-3 sec followed by 5 sec of drying under a stream of nitrogen. To functionalize the sensor surface, the carboxylic terminal groups of the binary SAM were converted to amine-reactive esters by applying 4 pl of a NHS/EDC (50 mM N-hydroxysuccinimide, 200 mM N-3-dimethylaminopropyl-N-ethylcarbodiimide, Sigma, St. Louis, Mo.) solution in deionized H₂O to the working electrode for 10 min. Activated sensors were incubated for 10 min with 4 pl of EZ-Link Amine-PEG₂-Biotin (Pierce, Rockford, Ill.) at a concentration of 5 mg/ml in 50 mM sodium acetate, pH 5. 30 μpl of 1M ethanolamine, pH 8.5 (Sigma, St. Louis, Mo.) was applied to all three electrodes for 10 min in order to block the remaining reactive groups of the activated monolayer. Biotinylated sensors were incubated in 4 μl of 0.5 mg/ml of streptavidin (Pierce) in RNase-free H₂O (Cat. No. 821739, MP Biomedicals, Aurora, Ohio) for 10 min. Streptavidin-coated sensors were incubated with biotinylated capture probes (4 μl, 1 μM in 1 M phosphate buffer, pH 7.2) for 30 min. Electrodes were blocked for 10 min with 4 μl of 0.05% polyethylene glycol 3350 (PEG, Sigma, St. Louis, Mo.) in 1 M phosphate buffer, pH 7.2. All these incubation steps were performed in a glass petri dish.

For both capture probe types, lysis of bacterial cells was performed by resuspending the appropriate pellet in 10 μL of 1 M NaOH and incubating at room temperature for 5 min. Bacterial lysates were neutralized by addition of 50 μl of 0.25 μM fluorescein (FITC)-modified detector probe in 1 M Phosphate Buffer pH 7.2 with 2.5% Bovine Serum Albumin (BSA) (Sigma, St. Louis, Mo.) and allowed to react for 10 min for homogeneous hybridization. Aliquots (4 μl) of this raw bacterial lysate target solution were cast onto each capture probe-modified sensor and incubated for 15 min. After washing and drying the array, 4 μl of a 0.5 U/ml anti-FITC horseradish peroxidase (HRP) Fab fragments (Roche, diluted in 0.5% casein in 1 M phosphate buffered saline, pH 7.2) solution were deposited on each of the working electrodes for 15 min. After washing and drying, a prefabricated plastic 16-well manifold (GeneFluidics, Irwindale, Calif.) was bonded to the sensor array. The sensor array was put into the chip reader and 50 μl of the TMB-H₂O₂ solution (Enhanced K-Blue TMB Substrate, Neogen, Lexington, Ky.) was placed on each of the sensors in the array, covering the three-electrode area. Chronoamperometric measurements were immediately and simultaneously taken for all 16 sensors by stepping the potential to −200 mV (vs the quasi Au reference electrode) and sampling the current at 60 s. For each array, negative control (NC) sensors were tested including the capture probe, FITC-detector probe, and the buffer (2.5% BSA in 1 M phosphate buffer, pH 7.2) instead of bacterial lysate solution. Positive controls (PC) were included in all sensor arrays and consisted of a synthetic target oligonucleotide for either the mature rRNA or Pre23S 3′Jxn pre-rRNA probe pairs at 1 nM with the corresponding detector probe (see Table 1).

Including the synthetic target molecule served to normalize the electrochemical signal intensity and determine the ribosomal and pre-ribosomal target molecule concentration because there is a linear log/log correlation between the concentration of the analyte and the electrochemical signal. The relation between the electrochemical signal generated and the number of synthetic target molecules tested were used to convert the electrochemical signal from samples at each time point into a number of target molecules per volume tested (concentration). This was then combined with the CFU/ml values determined by plating for each time point to generate target molecule number per CFU measurements.

Cryo-Electron Microscopy (Cryo-EM) of Frozen-Hydrated E. coli.

E. coli cultures (5 μl) were deposited onto a freshly glow discharged holey carbon grid, then blotted, and rapidly frozen in liquid ethane. The frozen-hydrated specimens were imaged at -170° C. using a Polara G2 electron microscope (FEI Company, Hillsboro, Oreg.) equipped with a field emission gun and a 4K×4K charge-coupled device (CCD; TVIPS, GMBH, Germany). The microscope was operated at 300 kV, and cryo-EM images were recorded at the magnification of 4,700×(3.76nm/pixel) and 31,000×(0.57nm/pixel), respectively. 9-14 cells were selected at random for length and width measurements at different times after inoculation.

### Results

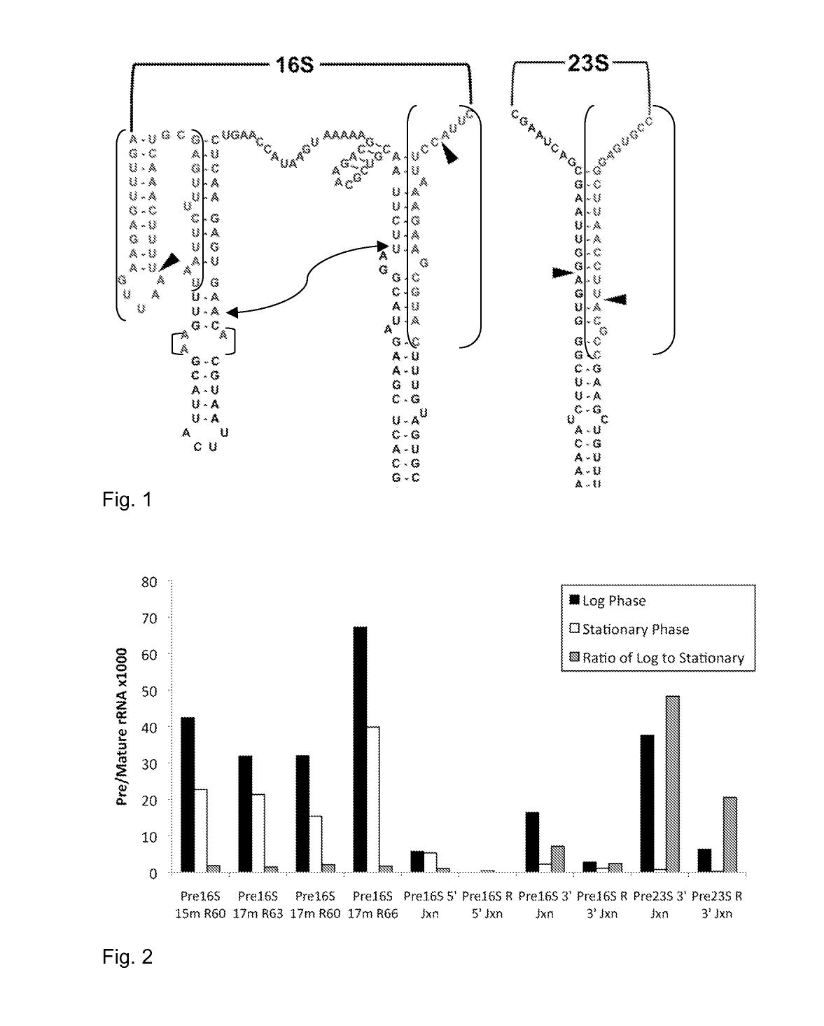

Development of Capture & Detector Probes for Pre-rRNA. Probe pairs were developed for pre-rRNA tails that are removed during rRNA processing (FIG. 1). Initially, probe pairs of various lengths were designed for a region in the 5′ tail of 16 S pre-rRNA predicted to be accessible for probe binding because it was relatively free of secondary structure. As shown in FIG. 2, some of these probe pairs demonstrated good sensitivity (high signal-to-noise ratio) for E. coli samples obtained during the log phase of growth. However, these 16S pre-rRNA probes generated unexpectedly low ratios of log phase to stationary phase signals when E. coli in different growth phases were tested (FIG. 2). These results indicated that such probes were not reliable markers for intact pre-rRNA molecules.

Subsequently, probe pairs were designed to hybridize with splice sites between the pre-rRNA tails and mature rRNA so that the target sequences would only be present in intact pre-rRNA (FIG. 1). These target sequences are digested into two pieces during processing of pre-rRNA into mature rRNA such that after digestion, neither piece of the target sequence would bind the probe sufficiently well to generate a signal. Probe pairs were tested for binding to the 5′ and 3′ splice sites of 16S rRNA and the 3′ splice site of 23S rRNA. Probe pairs in both the capture-detector and detector-capture orientations were tested. As shown in FIG. 2, pre-rRNA probe pairs targeting the splice sites resulted in higher ratios of log to stationary phase signals. These results are consistent with those of Cangelosi et al (3) who used a pre-rRNA sandwich hybridization assay in which their capture probe bound to the pre-rRNA tail and their detector probe bound to the mature rRNA region providing specificity for intact pre-rRNA. One of the two probe pairs for the 3′ splice site of 23S rRNA produced a high signal with a relatively high signal ratio for log phase compared to stationary phase cells. This capture (Pre23S 14m 3′JxnC) and detector (Pre23S 17m 3′JxnD) probe pair was selected for subsequent measurements of pre-rRNA.

For mature rRNA determination capture and FITC-detector probes specified in Table 1 were used.

Growth Phase Comparison of Mature rRNA vs. Pre-rRNA. We compared signals for mature rRNA vs. pre-rRNA for an overnight culture of E. coli before and after inoculation into fresh MH medium. Target rRNA and pre-rRNA concentrations were estimated by including known concentrations of synthetic artificial target oligonucleotides as internal calibration controls on each electrochemical sensor chip. These synthetic target oligonucleotides functioned by hybridizing with both the capture and detector probes. Copies per cell were calculated from the concentrations of the rRNA target number and the number of cells in the bacterial lysate. We found that variability in pre-rRNA and rRNA measurements could be reduced by chilling samples in an ice bath and centrifugation in a centrifuge refrigerated at 4° C. On the other hand, the cells were sensitive to cold shock particularly during the lag and early log phases of growth. For this reason, accurate plate counts were obtained by dilution of the culture in room temperature medium rather than cold medium.

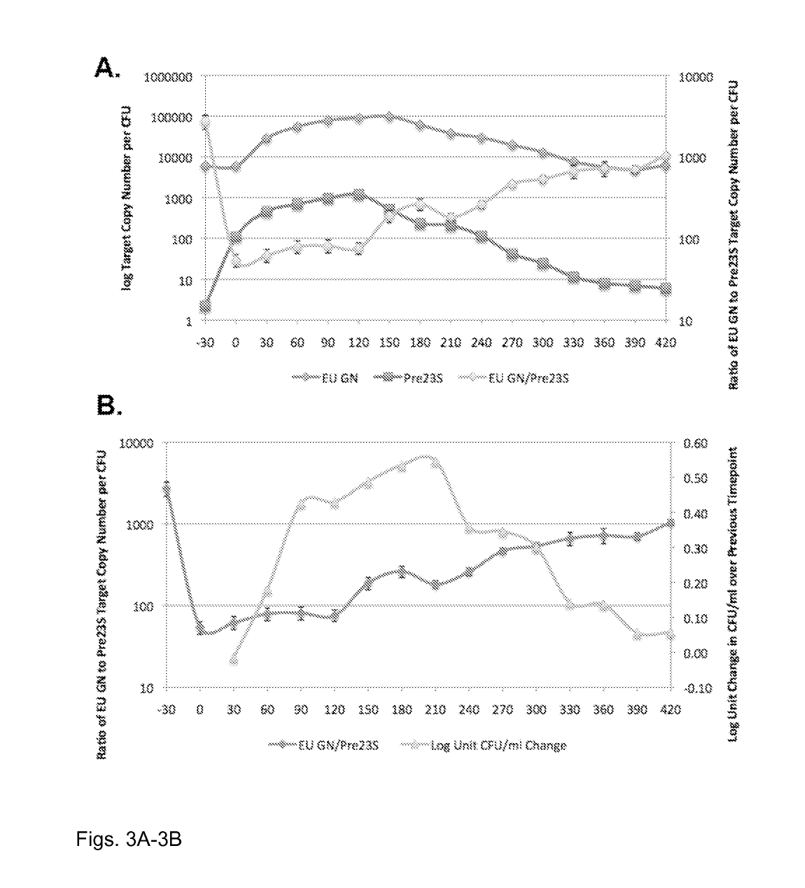

Immediately after inoculation of overnight culture into fresh growth medium (Table 2, time 0), there was a 55-fold increase in pre-rRNA from 2 copies per cell to 110 copies per cell, indicating a dramatic induction of rRNA synthesis. At that point, the ratio of mature rRNA to pre-rRNA reached a nadir of 54:1. As shown in FIG. 3, pre-rRNA levels continued to increase during the first two hours of incubation, peaking at 120 min after incubation at 1,200 copies per cell. As pre-rRNA was converted to mature rRNA, copies of mature rRNA peaked at >98,000 copies per cell at 150 min after inoculation. Despite a gradual drop in both mature rRNA and pre-rRNA thereafter, growth rate peaked at 210 min at 1.1 log unit increase in cellular concentration per hour, which equals a doubling time of 16.5 min. During the later phases of growth, pre-rRNA copy numbers dropped more quickly than mature rRNA copy numbers, eventually leading to an increase in the ratio of mature rRNA to pre-rRNA to >1000:1.

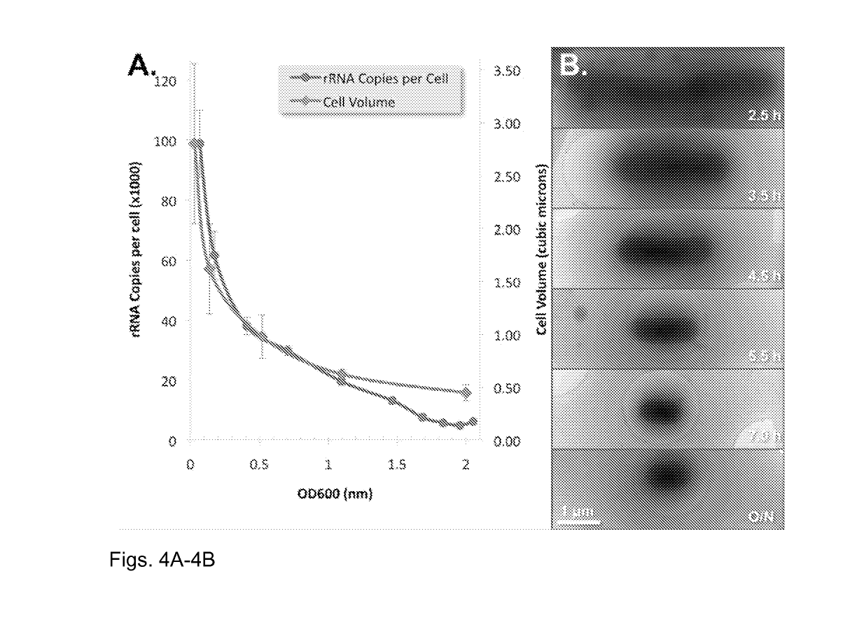

As shown in FIG. 4A, there was a good correlation between cell volume and rRNA copies per cell during log and late log phases of growth, indicating a relatively constant rRNA density in the cytoplasm. This correlation was lost at cell densities above OD_{600 nm}=1.0, at which point the cell volume stabilized while the rRNA copy number continued to fall. Cryo-electron microscopy was performed to measure E. coli cell volumes at different growth phases. As shown in FIG. 4B, E. coli cells became progressively shorter and thinner as cells went from log phase to stationary phase. The peak average cell size was 2.8 μm³ (4.87 μm long×0.85 μm wide) and the smallest average cell size was 0.45 μm³ (1.35 μm long×0.65 wide).

Effects of Antibiotics on Mature rRNA and Pre-rRNA Levels.

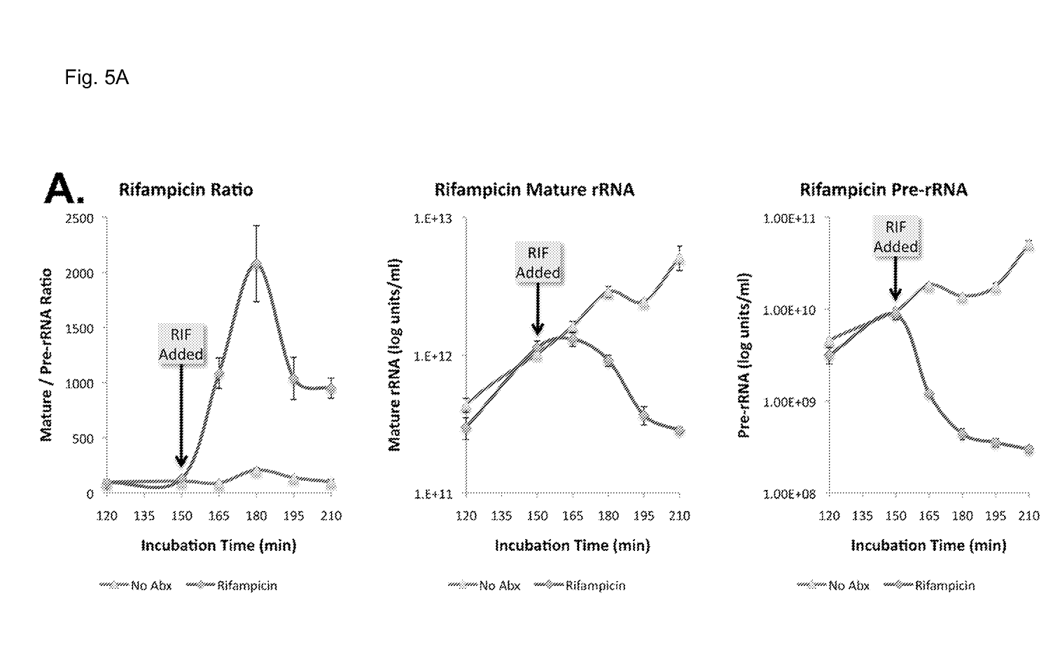

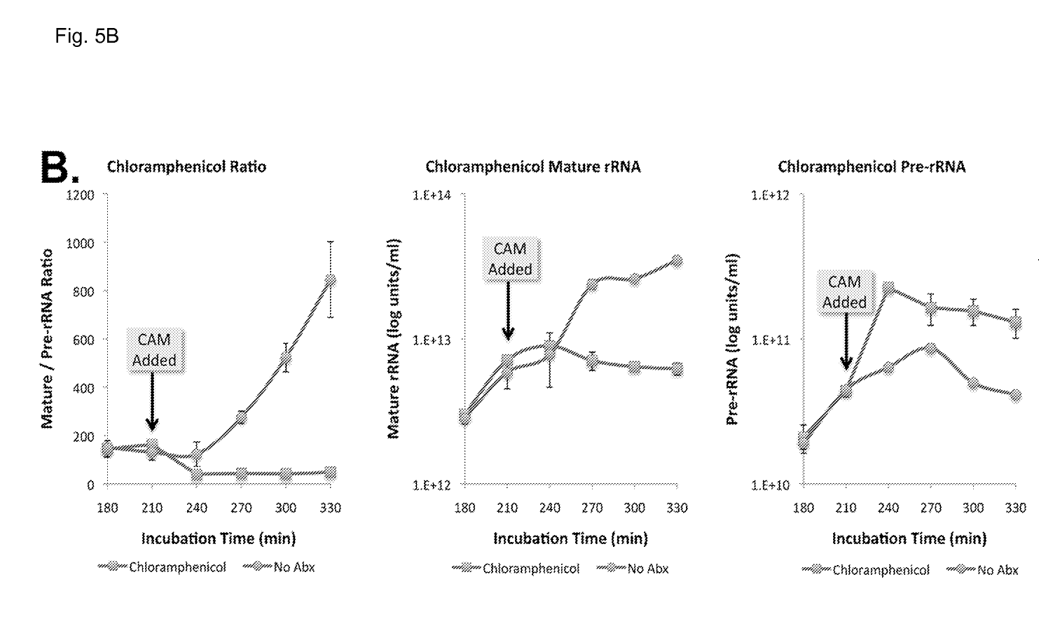

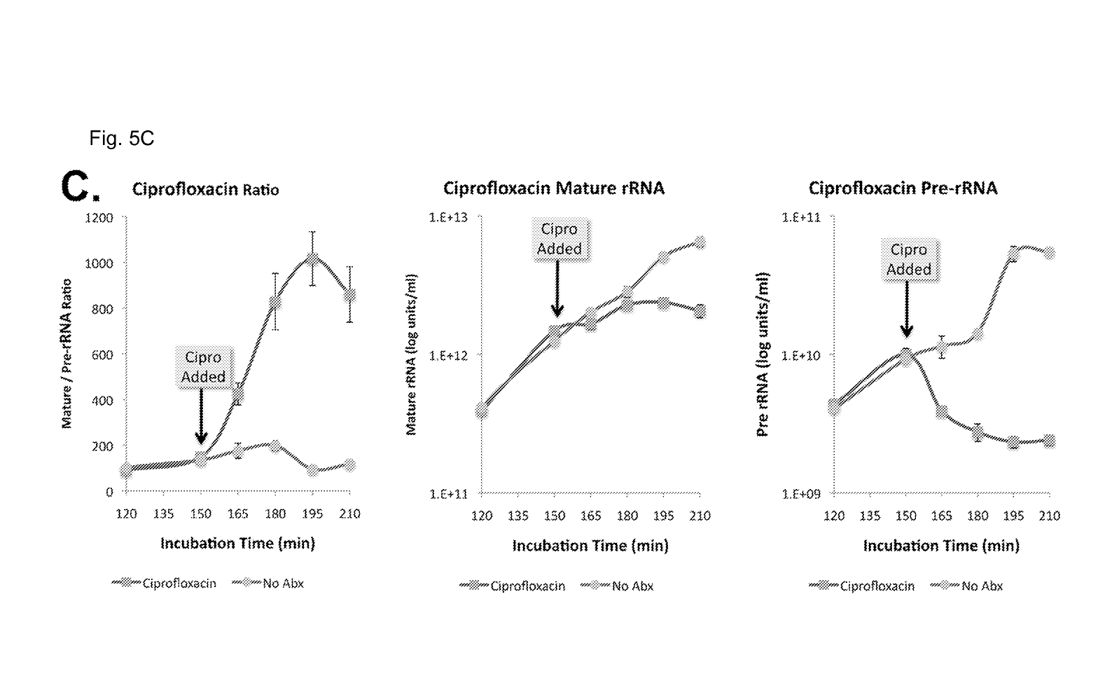

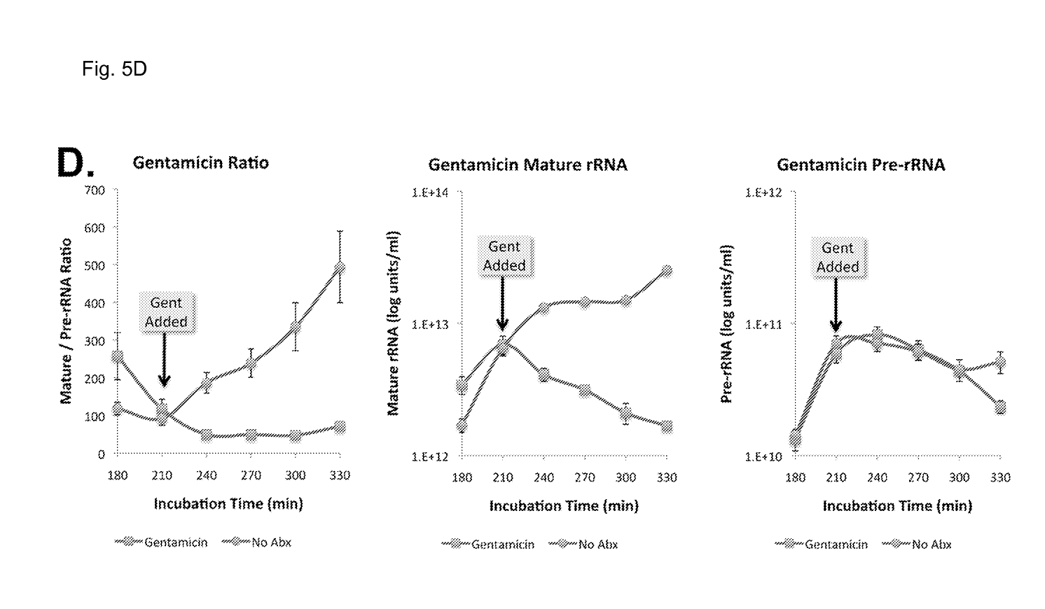

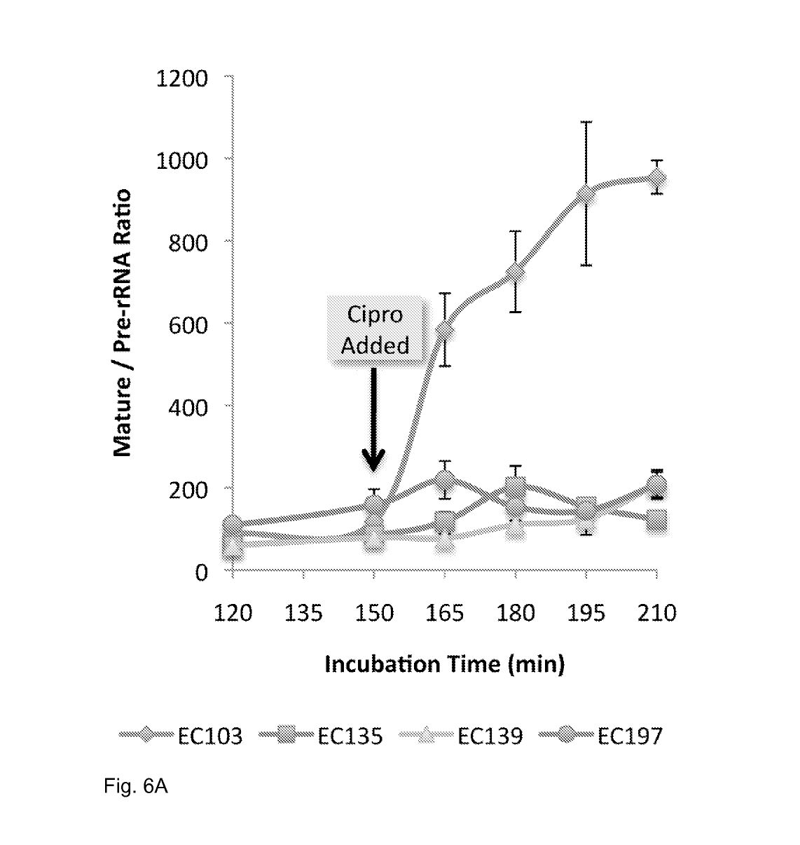

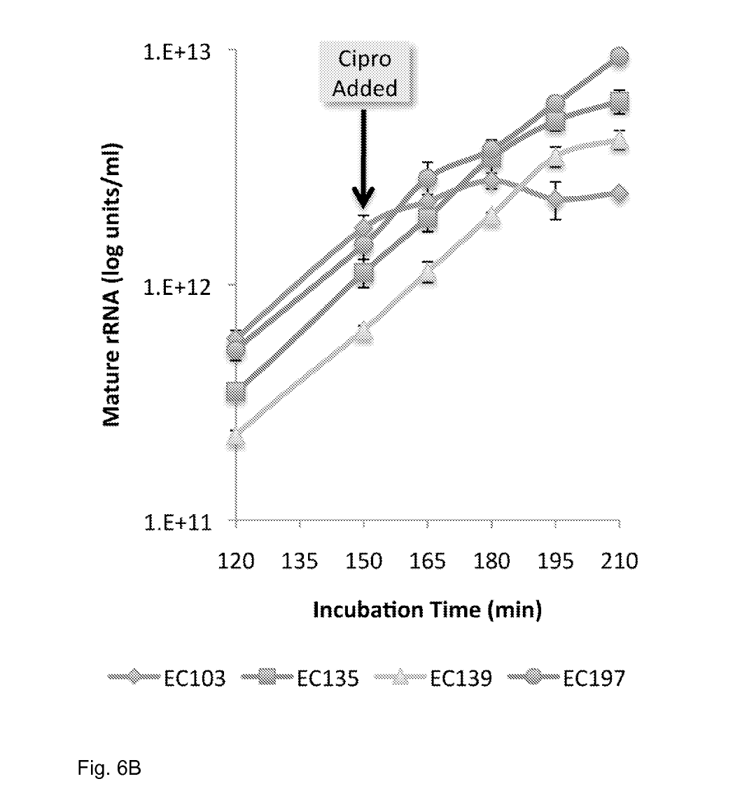

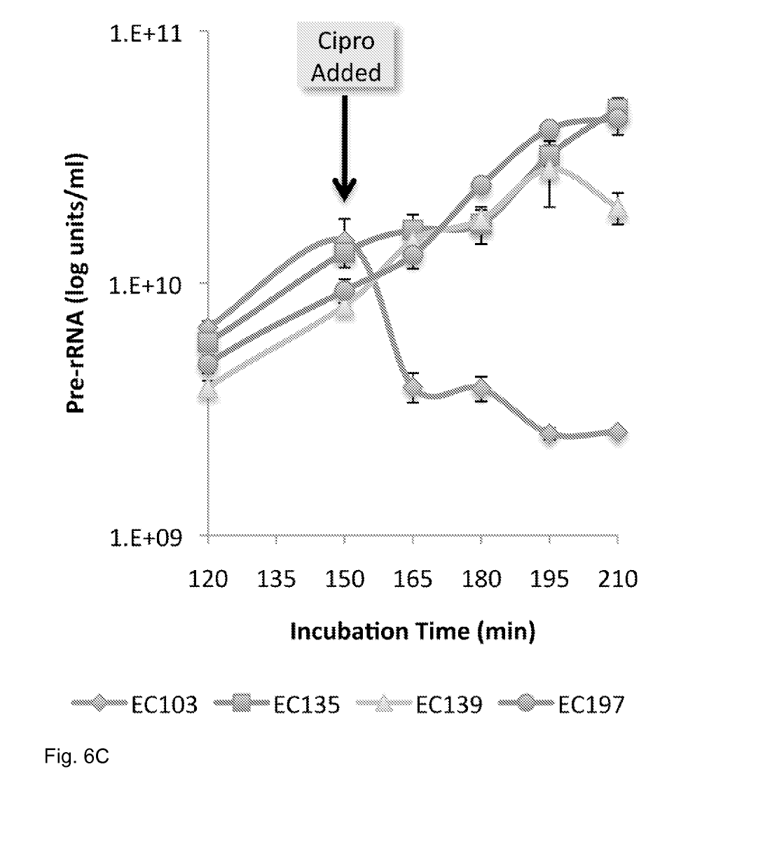

To confirm that the pre-rRNA capture and detector probes were selective for the desired target, we examined the effects of rifampicin and chloramphenicol on pre-rRNA levels relative to mature rRNA. Consistent with a previous report (3), addition of rifampicin caused a selective drop in pre-rRNA, while chloramphenicol caused a selective increase in pre-rRNA (FIGS. 5A & 5B). The effects of ciprofloxacin and gentamicin on pre-rRNA and mature rRNA levels were also examined. As shown in FIG. 5C, ciprofloxacin had an effect similar to that of rifampicin; pre-rRNA levels dropped significantly within 15 min while mature rRNA remained at control levels until 45 min after addition of the antibiotic. In contrast, there was no effect of this antibiotic on the pre-rRNA levels of ciprofloxacin-resistant organisms (FIG. 6). Addition of gentamicin resulted in a decrease in mature rRNA without affecting the level of pre-rRNA (FIG. 5D).

TABLE 1
DNA oligonucleotides used in this study

| | | SEQ |
|---|---|---|
| | | ID |
| Probe Name¹ | Sequence² | NO: |
| Pre16S 15m 48D | 5′-TTTTTCGTCTTGCGA-F | 13 |
| Pre16S 15m 63C | 5′-B-GAGACTTGGTATTCA | 14 |
| Pre16S 15m R63D | 5′-F-GAGACTTGGTATTCA | 15 |
| Pre16S 15m R48C | 5′-TTTTTCGTCTTGCGA-B | 16 |
| Pre16S 17m R63D | 5′-F-TTGAGACTTGGTATTCA | 17 |
| Pre16S 17m R46C | 5′-TTTTTCGTCTTGCGACG-B | 18 |
| Pre16S 19m R63D | 5′-F-TCTTGAGACTTGGTATTCA | 19 |
| Pre16S 19m R44C | 5′-TTTTTCGTCTTGCGACGTT-B | 20 |
| Pre16S 21m R63D | 5′-F-ACTCTTGAGACTTGGTATTCA | 21 |
| Pre16S 21m R42C | 5′-TTTTTCGTCTTGCGACGTTAA-B | 22 |
| Pre16S 17m R60D | 5′-F-AGACTTGGTATTCATTT | 23 |
| Pre16S 17m R43C | 5′-TTCGTCTTGCGACGTTA-B | 24 |
| Pre16S 19m R60D | 5′-F-TGAGACTTGGTATTCATTT | 25 |
| Pre16S 19m R41C | 5′-TTCGTCTTGCGACGTTAAG-B | 26 |
| Pre16S 21m R60D | 5′-F-CTTGAGACTTGGTATTCATTT | 27 |
| Pre16S 21m R39C | 5′-TTCGTCTTGCGACGTTAAGAA-B | 28 |
| Pre16S 17m R66D | 5′-F-CTCTTGAGACTTGGTAT | 29 |
| Pre16S 17m R49C | 5′-TCATTTTTCGTCTTGCG-B | 30 |
| Pre16S 19m R66D | 5′-F-CACTCTTGAGACTTGGTAT | 31 |
| Pre16S 19m R47C | 5′-TCATTTTTCGTCTTGCGAC-B | 32 |
| Pre16S 21m R66D | 5′-F-TTCACTCTTGAGACTTGGTAT | 33 |
| Pre16S 21m R45C | 5′-TCATTTTTCGTCTTGCGACGT-B | 34 |
| Pre16S 19m 5′JxnD | 5′-TTTGATGCTCAAAGAATTA-F | 35 |
| Pre16S 21m 5′JxnC | 5-S-TCAAACTCTTCAATTTAAAAG | 36 |
| Pre16S 21m R5′JxnD | 5-F-TCAAACTCTTCAATTTAAAAG | 37 |
| Pre16S 19m R5′JxnC | 5′-TTTGATGCTCAAAGAATTA-S | 38 |
| Pre16S 17m 3′JxnD | 5′-GAGGTGATCCAACCGCA-F | 39 |
| Pre16S 20m 3′JxnC | 5-S-GAACGCTTCTTTAAGGTAAG | 40 |
| Pre16S 20m R3′JxnD | 5-F-GAACGCTTCTTTAAGGTAAG | 41 |
| Pre16S 17m R3′JxnC | 5′-GAGGTGATCCAACCGCA-S | 42 |
| *Pre23S 17m 3′JxnD | 5′-AAGCCTCACGGTTCATT-F | 5 |
| *Pre23S 14m 3′JxnC | 5′-S-GGCGTTGTAAGGTT | 6 |
| Pre23S 14m R3′JxnD | 5′-F-GGCGTTGTAAGGTT | 43 |
| Pre23S 17m R3′JxnC | 5′-AAGCCTCACGGTTCATT-S | 44 |
| Mature rRNA 18m | 5′-GTTACGACTTCACCCCAG-F | 45 |
| 1484D | | |
| Mature rRNA 19m | 5′-S-GTTCCCCTACGGTTACCTT | 46 |
| 1502C | | |

| | |
|---|---|
| Synthetic Target Oligonucleotides: | |
| Pre-rRNA 31m 5′- | 47 |
| AATGAACCGTGAGGCTTAACCTTACAACGCC | |
| Mature rRNA 37m 5′- | 48 |
| CTGGGGTGAAGTCGTAACAAGGTAACCGTAGGGGAAC | |

¹Abbreviations: Number of nucleotides (m), capture probe (C), detector probe (D), splice site (Jxn), reverse orientation (R).
²Abbreviations: FITC (F), biotin (B), thiol (S).
*Indicates capture & detector probe pair selected based on its high signal-to-noise ratio.

TABLE 2
Pre- and mature rRNA quantitation during E. coli growth phases.

| Timepoint | | | | | Gen. | | pre- | | GROWTH |
|---|---|---|---|---|---|---|---|---|---|
| (min) | OD₆₀₀ | CFU/ml | Rate^{a} | Doublings^{b} | time^{c} | rRNA^{d} | rRNA^{d} | Ratio | PHASE |
| Overnight | 2.307 | 6.67E+09 | — | — | — | 6,009 | 2 | 2,720 | STATIONARY |
| Culture | | | | | | | | | PHASE |
| 0 | 0 | 5.90E+05 | — | — | — | 5,942 | 110 | 54 | LAG PHASE |
| 30 | 0 | 5.70E+05 | — | — | — | 28,427 | 457 | 62 | |
| 60 | 0.003 | 8.63E+05 | 0.18 | 1.20 | 50.08 | 55,278 | 696 | 79 | LOG PHASE |
| 90 | 0.008 | 2.30E+06 | 0.43 | 2.83 | 21.19 | 78,741 | 970 | 81 | |
| 120 | 0.024 | 6.20E+06 | 0.43 | 2.86 | 21.00 | 91,049 | 1200 | 76 | |
| 150 | 0.068 | 1.91E+07 | 0.49 | 3.24 | 18.51 | 98,782 | 523 | 189 | |
| 180 | 0.175 | 6.53E+07 | 0.53 | 3.55 | 16.88 | 61,571 | 235 | 262 | |
| 210 | 0.409 | 2.30E+08 | 0.55 | 3.64 | 16.50 | 38,033 | 208 | 183 | |
| 240 | 0.707 | 5.27E+08 | 0.36 | 2.39 | 25.14 | 29,801 | 114 | 260 | EARLY |
| 270 | 1.095 | 1.17E+09 | 0.35 | 2.30 | 26.14 | 19,610 | 42 | 466 | STATIONARY |
| 300 | 1.466 | 2.33E+09 | 0.30 | 2.00 | 30.00 | 13,162 | 25 | 536 | PHASE |
| 330 | 1.686 | 3.23E+09 | 0.14 | 0.94 | 63.74 | 7,608 | 11 | 665 | STATIONARY |
| 360 | 1.834 | 4.43E+09 | 0.14 | 0.91 | 65.87 | 5,734 | 8 | 723 | PHASE |
| 390 | 1.957 | 5.03E+09 | 0.06 | 0.37 | 163.81 | 4,899 | 7 | 706 | |
| 420 | 2.051 | 5.73E+09 | 0.06 | 0.38 | 159.68 | 6,230 | 6 | 1,049 | |

^{a}Growth rate in log units per 30 min.
^{b}Doublings per hour.
^{c}Generation time (min).
^{d}Copies per cell.

### Discussion

We describe an electrochemical sensor assay for detection and quantitation of pre-rRNA. Pre-rRNA represents a labile pool of rRNA precursor molecules produced during rRNA transcription. Pre-rRNA differs from mature rRNA by the presence of 5′ and 3′ tails that are removed during the maturation process. Because pre-rRNA represents a relatively small fraction (0.1%-10%) of total rRNA, a sensitive assay is required for its detection. To achieve the needed sensitivity, our electrochemical Au-sensor assay relies on the use of a ternary interface involving hexanedithiol co-immobilized with a thiolated capture probe, followed by the incorporation of 6-mercapto-1-hexanol as diluent. This new interface has been shown to offer a greatly improved surface blocking and maximal hybridization efficiency allowing ultrasensitive electrochemical detection of target nucleic acids (2, 8, 17). Direct nucleic acid detection methods, such as the electrochemical sandwich hybridization assay described herein, have inherent advantages over methods that require target amplification, such as qRT-PCR. We were able to quantitate pre-rRNA during different E. coli growth phases, and documented dramatic shifts in copy number from 2 to 1,200 copies per cell in the stationary and log phases of growth, respectively. This is the first time that pre-rRNA copy numbers per cell have been quantitated electrochemically. The 600-fold increase in pre-rRNA copy number that we observed is considerably larger than the 50-fold increase reported by Cangelosi et al (3) using luminescence detection. Possible reasons for this difference include a low limit of detection and the E. coli strain type. Cangelosi et al examined the E. coli type strain ATCC 11775, which was isolated in 1895 by Migula, and may have undergone metabolic changes during passage. In contrast, our studies were performed on a recently isolated wild-type uropathogenic E. coli strain with a relatively fast peak doubling time of 16.5 minutes.

Antibiotics differ in their effects on pre-rRNA and mature rRNA. Rifampicin is an inhibitor of prokaryotic DNA-dependent RNA polymerase. Because pre-rRNA is rapidly processed to mature rRNA, inhibiting transcription quickly reduces the pool of pre-rRNA, especially during the log phase of growth. In contrast, chloramphenicol and gentamicin are protein synthesis inhibitors. Chloramphenicol acts by binding to the 23 S subunit of bacterial ribosomes to inhibit protein synthesis, whereas gentamicin acts by inhibiting the proof-reading function of ribosomes, thereby introducing translation errors and premature peptide chain termination events. In either case, these protein synthesis inhibitors should not directly interfere with pre-rRNA synthesis. Accordingly, we observed a decrease in the pool of mature rRNA, presumably because of the loss of proteins required for ribosome formation and stability. In the case of chloramphenicol, inhibition of pre-rRNA processing not only resulted in a decrease of mature rRNA but an increase in pre-rRNA (FIG. 5B).

Ciprofloxacin is a quinolone antibiotic that inhibits the activity of DNA gyrase, the bacterial topoisomerase that introduces and relaxes DNA supercoils. Relaxing of supercoils is required for unpackaging of DNA prior to not only DNA replication but also RNA transcription (16). As in the case of rifampicin, inhibition of RNA transcription by ciprofloxacin resulted in a rapid decrease in pre-rRNA, detectable within fifteen minutes after addition of the antibiotic. Quinolone resistance typically results from gyrase mutations that prevent binding of the quinolone to the gyrase. As expected, addition of ciprofloxacin had no discernable effect of pre-rRNA levels in ciprofloxacin resistant organisms (FIG. 6).

There is a considerable interest in methods for determining the susceptibility of bacteria in clinical specimens in a time frame sufficient to impact clinical decision making. A major drawback of current clinical bacteriology methods is the need to isolate bacteria on solid agar media when processing a clinical specimen. In the absence of expeditious antibiotic susceptibility testing, clinicians typically initiate “empiric” antibiotic treatment, meaning that antibiotics are chosen based on prior knowledge of potential organisms and their antibiotic resistance patterns. Empiric antibiotics for bacteremia are typically broad-spectrum to treat a wide variety of possible bacterial pathogens. This approach is especially problematic in the management of complex urinary tract infections where quinolone-resistance rates are typically 20-30% (5). In addition, overuse of broad-spectrum antibiotics contributes to the emergence of antibiotic resistance by applying selective pressure to the patient's flora and favoring colonization by resistant organisms.

To address the need for antibiotic resistance data at the time of initial antibiotic selection, methods are needed to analyze the antibiotic susceptibility of organisms in clinical specimens. The electrochemical sensor assay has been validated on human clinical urine specimens from patients with urinary tract infection (9, 11). Electrochemical sensor assays for pre-rRNA would be expected to be useful for identifying bacteria that are susceptible to antibiotics such as rifampicin and ciprofloxacin that directly or indirectly inhibit RNA transcription. It may be possible to extend this approach for antibiotic susceptibility testing to other drugs by first depleting pre-rRNA levels and then measuring the ability of the antibiotic to inhibit pre-rRNA replenishment (4). However, because antibiotics act by widely divergent mechanisms, various approaches may be necessary to achieve comprehensive antibiotic susceptibility testing. For example, we have successfully applied ATP bioluminescence to determine antimicrobial susceptibility of uropathogens within 120 min after inoculation of clinical urine specimens into growth medium with and without antibiotics (7). Application of such assays to bacteria in clinical specimens at the point of care would enable patient-specific antibiotic therapy.

### Example 2

### Use of Pre-rRNA to Assess Growth Phase of Bacteria

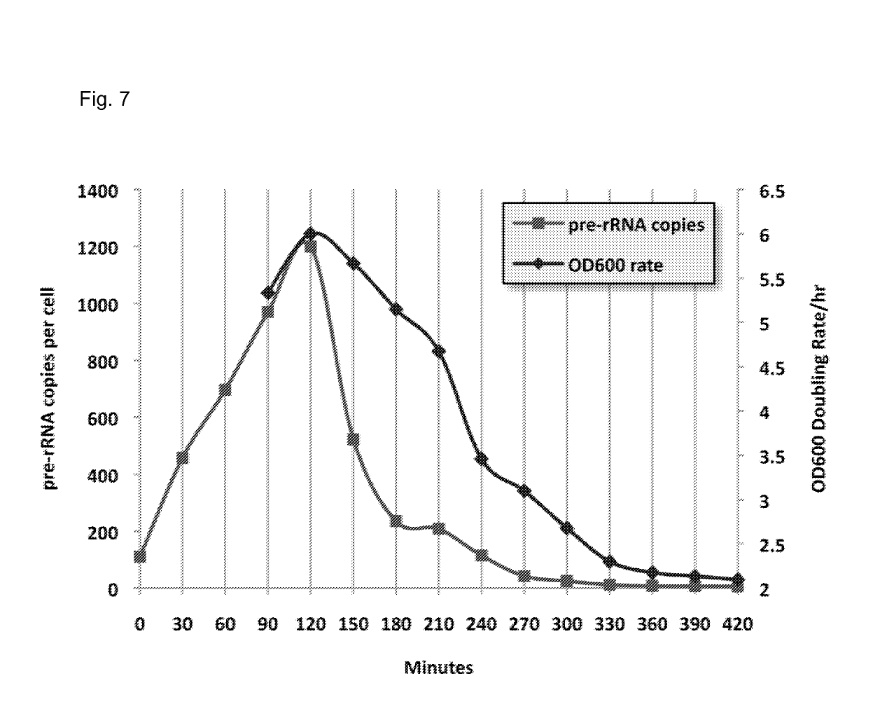

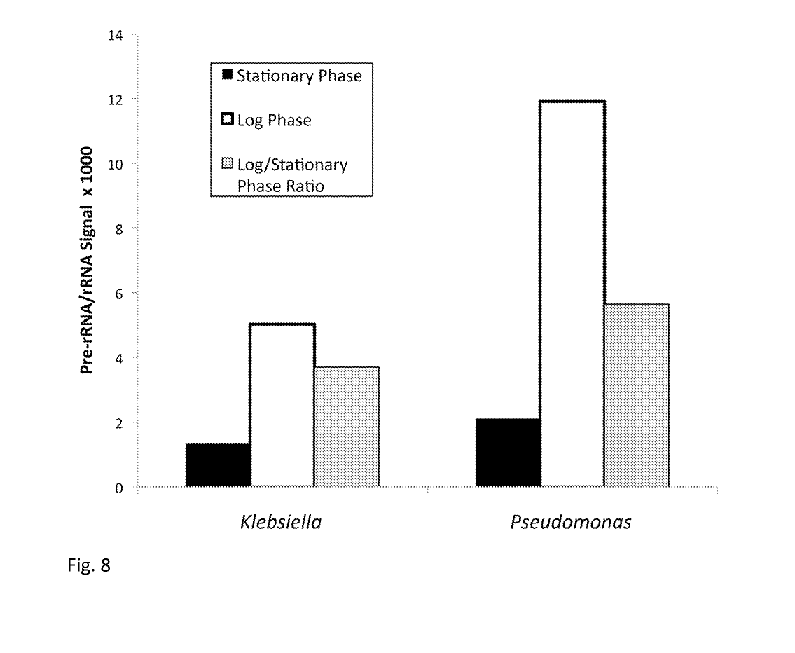

The correlation between pre-rRNA copies per cell and bacterial growth rate is depicted in FIG. 7. Growth rate is based on total cell volume as measured by turbidity or the increase in optical density at 600 nm, which peaks at 120 minutes, the same time as the peak in number of prRNA copies per cell. FIG. 8 illustrates the evaluation of pre-rRNA probe pairs in gram-negative bacteria. The ratio of signals from probe pairs specific for pre-rRNA to mature rRNA were compared in overnight (O/N) or stationary phase cultures and in cultures in the log phase of growth. Pre-rRNA signals were four-fold higher in log phase

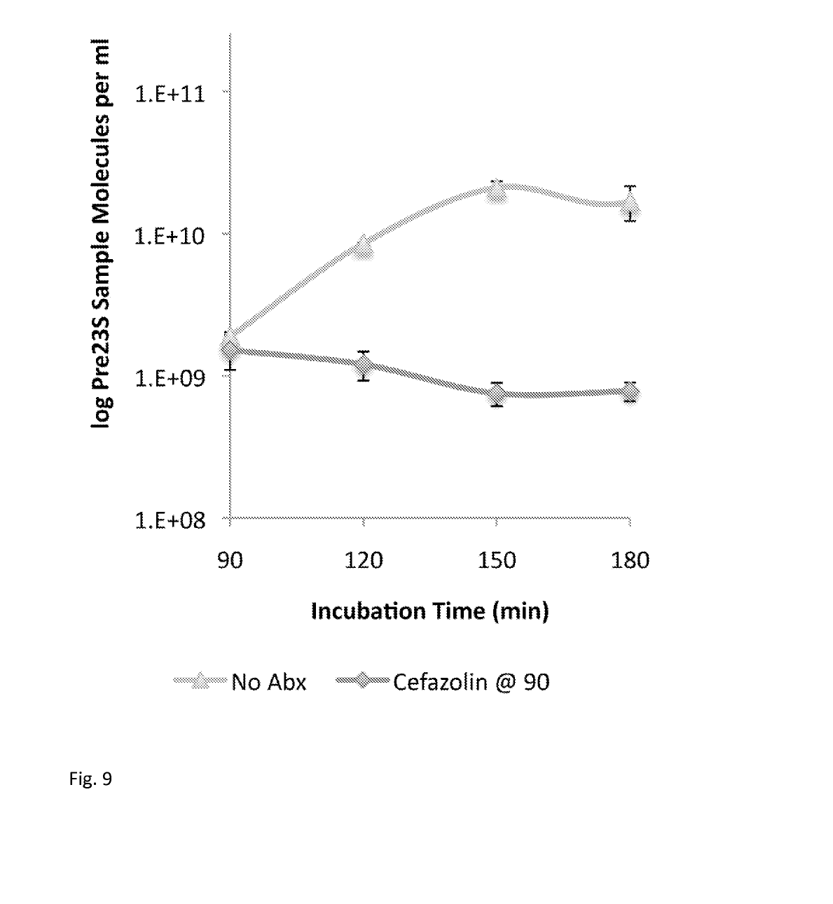

Klebsiella cells than in stationary phase Klebsiella cells, and six-fold higher in log phase Pseudomonas cells than in stationary phase Pseudomonas cells. FIG. 9 shows the response of pre-rRNA to cefazolin. Addition of cefazolin, a beta-lactam antibiotic, to a culture of a susceptible strain of E. coli in the log phase of growth resulted in a one-log drop in the amount pre-rRNA within 30 min compared to a culture without the antibiotic. Error bars estimated the standard deviation.

### REFERENCES

1. Bremner, H., and P. P. Dennis. 1996. Modulation of chemical composition and other parameters of the cell by growth rate, p. 1553-1569. In F. C. Neidhardt (ed.), Escherichia coli and Salmonella, vol. 2. ASM Press, Washington, D. C.
2. Campuzano, S., et al. 2011. Biosens Bioelectron 26:3577-3584.
3. Cangelosi, G. A., and W. H. Barbant. 1997. J Bacteriol 179:4457-4463.
4. Cangelosi, G. A., et al. 1996. Antimicrob Agents Chemother 40:1790-1795.
5. Cullen, et al. 2011. Br J Urol Int [Epub ahead of print].
6. Fuchs, B. M., et al. 1998. Appl Environ Microbiol 64:4973-4982.
7. Ivancic, V., et al. 2008. J Clin Microbiol 46:1213-1219.
8. Kuralay, F., et al. 2011. Talanta 85:1330-1337.
9. Liao, J. C., et al. 2006. J Clin Microbiol 44:561-570.
10. Liao, J. C., et al. 2007. J Mol Diagn 9:158-168.
11. Mach, K. E., et al. 2009. J Urol 182:2735-2741.
12. Mastali, M., et al. 2008. J Clin Microbiol 46:2707-2716.
13. Sun, C. P., et al. 2005. Mol Genet Metab 84:90-99.
14. Wang, J. 2006. Analytical Electrochemistry. J. Wiley, New York.
15. Wang, J. 2008. Electrochemical glucose biosensors. Chem Rev 108:814-825.
16. Willmott, C. J., et al. 1994. J Mol Biol 242:351-363.
17. Wu, J., et al. 2010. Anal Chem 82:8830-8837.
18. Wu, J., et al. 2009. Anal Chem 81:10007-10012.

### Example 3

### Amdinocillin for Rapid Determination of Susceptibility to Beta-lactam Antibiotics

To rapidly detect susceptibility to the quinolone antibiotic, ciprofloxacin, it was necessary to measure levels of precursor-rRNA (pre-rRNA) levels instead of mature rRNA. In this study, we examined beta-lactam antibiotics for their effects on mature rRNA and pre-rRNA. Bacteria are unable to divide but may continue to grow in length for a period of time in the presence of beta-lactam antibiotics to which they are susceptible. The process of growth in length without division is called filamentation. Beta-lactam antibiotics that bind preferentially to penicillin-binding protein (PBP) 3 are more likely to cause filamentation. Filamentation may continue for more than an hour until a cell lysis event related to rupture of the cell wall. During filamentation, cellular mature rRNA and pre-rRNA increases, delaying the ability of rRNA- or pre-rRNA-based assays to differentiate susceptible from resistant bacteria.

We found that the PBP2-binding compound, amdinocillin (also known as mecillinam), prevents filamentation caused by beta-lactam antibiotics. As a result of blocking filamentation, levels of mature rRNA and pre-rRNA decrease within 30-45 min when amdinocillin is combined with beta lactam antibiotics. For some beta-lactam antibiotics, such as ampicillin, the amdinocillin effect is independent of the antibiotic concentration. For other antibiotics, such as cefazolin and imipenem, the amdinocillin effect is dependent on the antibiotic concentration. For example, at a concentration of 32 μg/ml, cefazolin causes a drop of pre-rRNA within 30 min whether or not amdinocillin is present. In contrast, at a concentration of 4 μg/ml, cefazolin does not cause a drop of pre-rRNA unless amdinocillin is present.

The amdinocillin effect has been demonstrated for antibiotics belonging to three major classes of beta-lactam antibiotics: penicillins, cephalosporins, and carbapenems. Addition of amdinocillin to antibiotic-susceptibility assays involving ampicillin, piperacillin, cefazolin, cefotaxime, and imipenem enables rapid differentiation of antibiotic-susceptible from antibiotic-resistant bacteria. Importantly, amdinocillin alone had no effect on rRNA or pre-rRNA levels. Furthermore, disc diffusion studies demonstrated that amdinocillin did not increase the susceptibility of bacteria to beta-lactam antibiotics. In other words, the amdinocillin does not cause antibiotic-resistant bacteria to appear susceptible to beta-lactam antibiotics. These are the first studies demonstrating that a PBP2-specific compound enables rapid determination of antibiotic susceptibility.

Graphs depicting effects of ampicillin with or without amdinocillin on mature rRNA and pre-rRNA are shown in FIGS. 11A-11C. Ampicillin-susceptible E. coli (strain EC135) was treated with ampicillin (16 μg/ml) alone, amdinocillin (1 μg/ml) alone, ampicillin plus amdinocillin, or neither. Amdinocillin enabled early recognition of ampicillin susceptibility. Effects on pre-rRNA were more pronounced than those on mature rRNA.

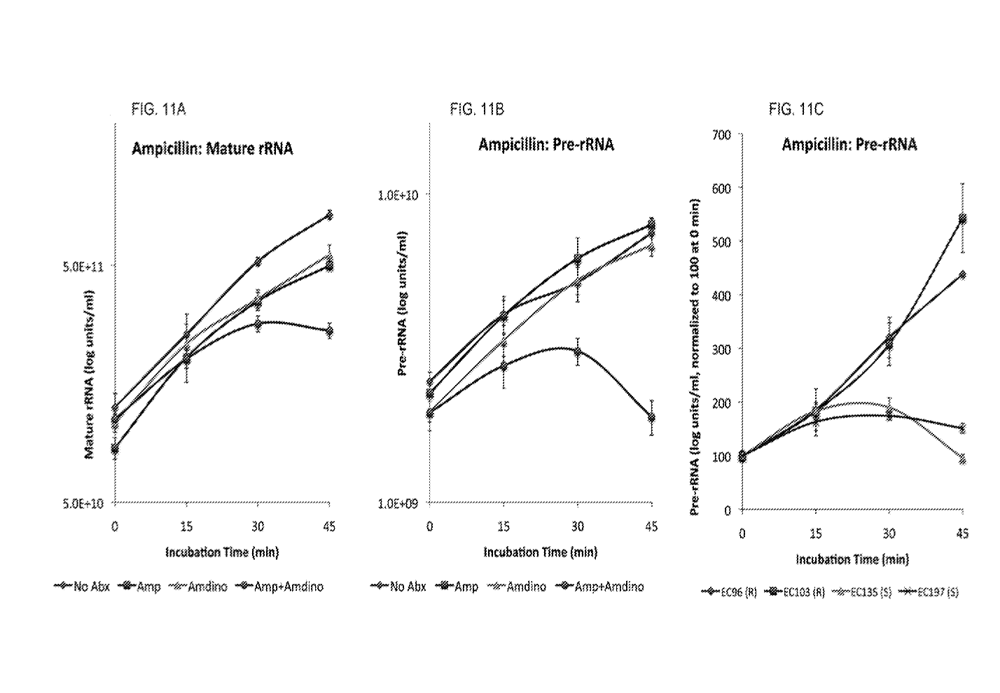

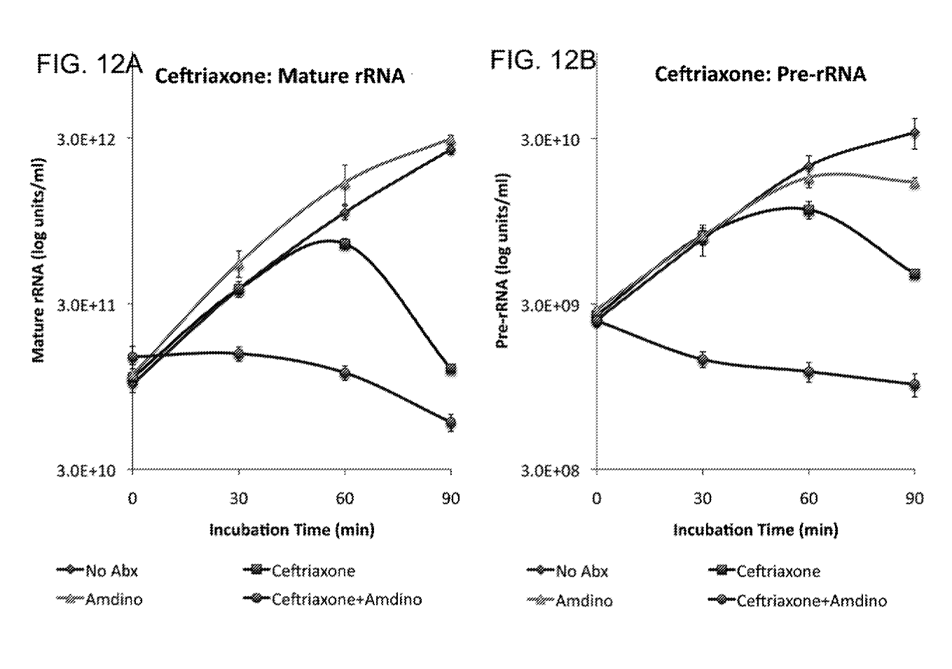

Graphs illustrating effects of ceftriaxone with our without amdinocillin on mature rRNA and pre-rRNA are presented in FIGS. 12A-12B. Ceftriaxone-susceptible E. coli (strain EC103) was treated with ceftriaxone (8 pg/ml) alone, amdinocillin (1 μ/ml) alone, ceftriaxone plus amdinocillin, or neither. Ceftriaxone-mediated effects on mature rRNA and pre-rRNA occurred 60 min faster with amdinocillin than without amdinocillin.

Graphs showing that effects on pre-rRNA are concentration dependent are shown in FIGS. 13A-13C. Cefazolin-susceptible E. coli (strain EC103) was treated with amdinocillin (1 μg/ml) plus cefazolin at concentrations ranging from 0-32 μg/ml. At a cefazolin concentration of 32 μg/ml, amdinocillin had no effect on pre-RNA levels. At a cefazolin concentration of 4 μg/ml, amdinocillin enabled early recognition of cefazolin-susceptibility. Pre-rRNA levels fell more quickly at higher cefazolin concentrations.

Graphs illustrating the effects of beta-lactam antibiotics plus amdinocillin on antibiotic susceptible and resistant bacteria can be found in FIGS. 14A-14D. Antibiotic susceptible and resistant bacteria were treated with amdinocillin (1 μg/ml) plus various beta lactam antibiotics including 16 μg/ml cefazolin, 4 μg/ml ceftriaxone, 32 μg/ml piperacillin plus 4 μg/ml tazobactam, and 2 μg/ml imipenem. Differential effects on pre-rRNA of susceptible from resistant bacteria were evident within 30-90 min.

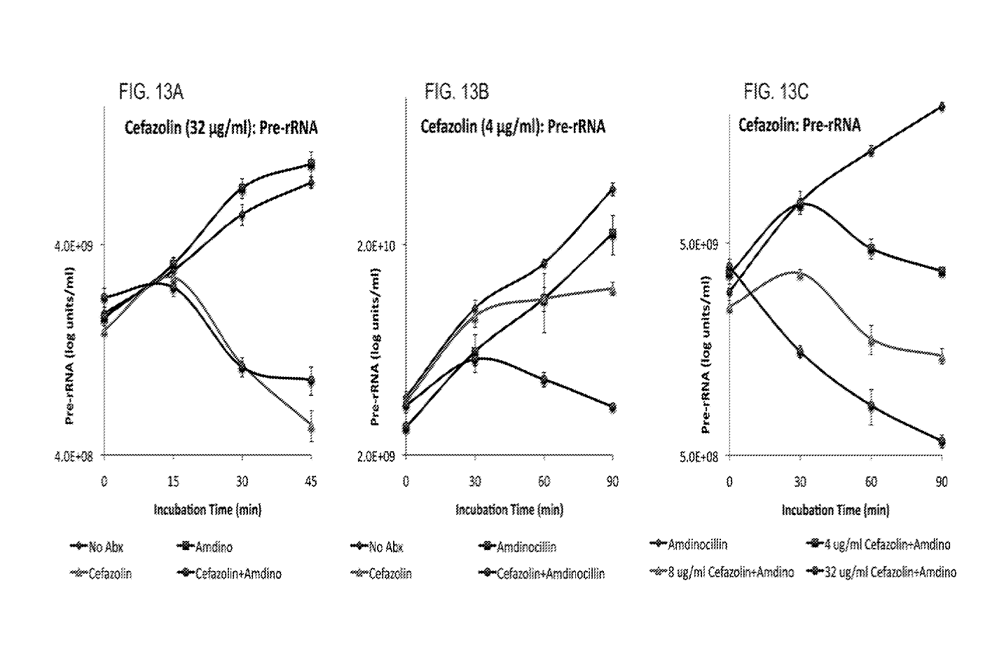

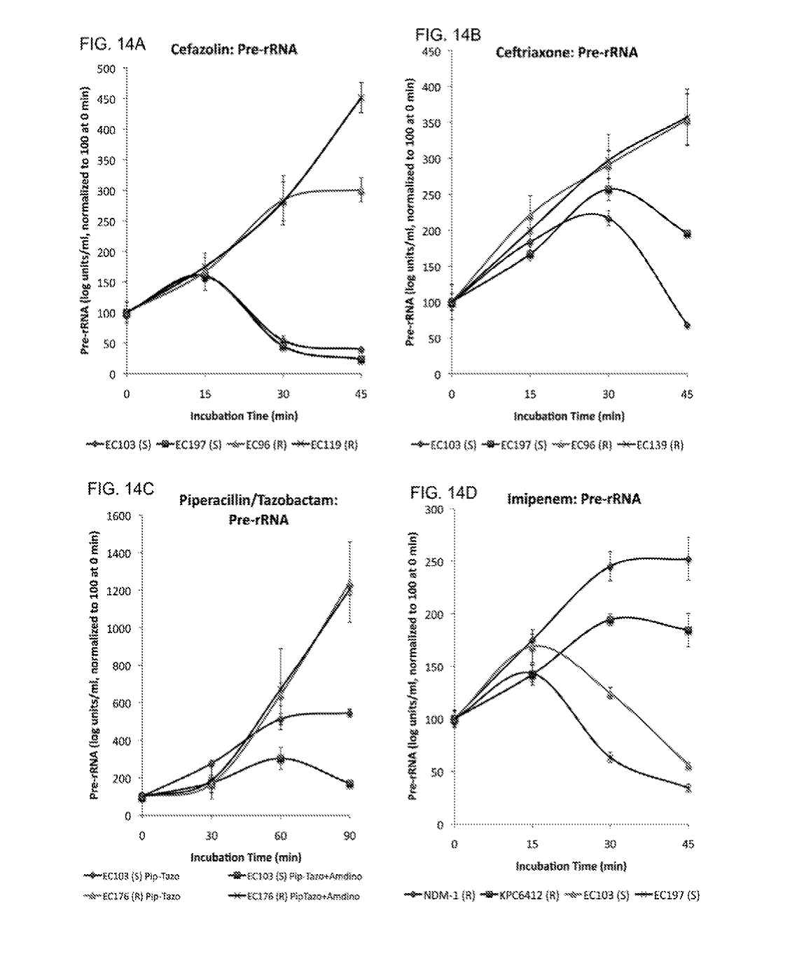

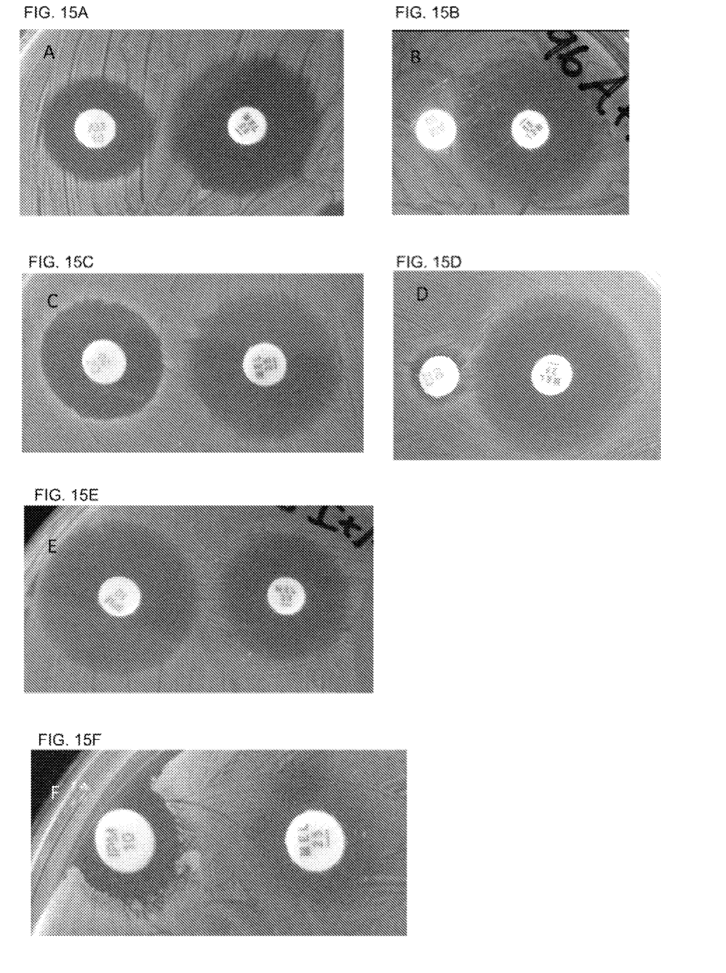

FIGS. 15A-15F present digital photomicrographs showing results of Kirby-Bauer disc diffusion antibiotic-susceptibility tests. 6 mm diameter antibiotic discs were placed on agar plates seeded with a lawn of E. coli that were Ampicillin susceptible (strain EC135), Ampicillin resistant (strain EC96), Cefazolin susceptible (strain EC103), Cefazolin resistant (strain EC96), Imipenem susceptible (strain EC103) or Imipenem resistant (strain NDM-1). In each panel the antibiotic disc is on the left and the amdinocillin disc is on the right. Images were obtained after 20 hours of incubation at 37° C. Synergy was not observed between amdinocillin and any of the antibiotics tested.

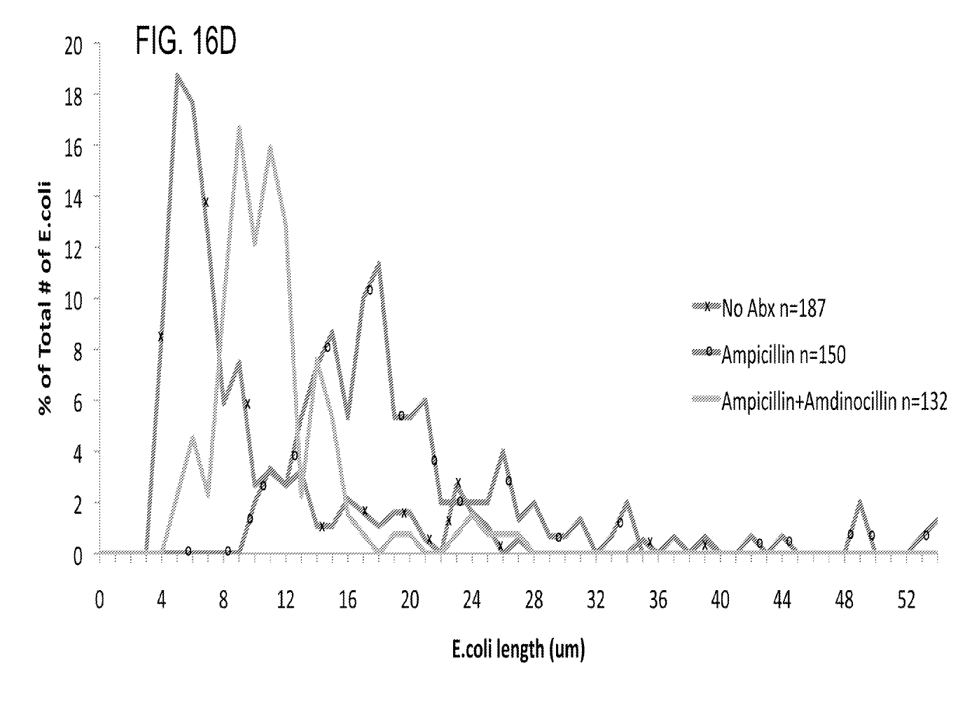

The data shown in FIGS. 16A-16D demonstrate that amdinocillin blocks ampicillin-induced E. coli filamentation. The lengths of ampicillin-susceptible E. coli (strain EC103) cells were measured after treatment for 30 minutes with no antibiotics, ampicillin, or ampicillin plus amdinocillin. Digital photomicrographs of representative cells treated with no antibiotics (16A), ampicillin (16B), and ampicillin plus amdinocillin (16C) are shown. FIG. 16D is a frequency histogram showing that ampicillin caused an average 4-fold increase in length of E. coli cells, which was partially blocked by amdinocillin.

Those skilled in the art will appreciate that the conceptions and specific embodiments disclosed in the foregoing description may be readily utilized as a basis for modifying or designing other embodiments for carrying out the same purposes of the present invention. While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for determining whether a sample of bacteria is susceptible to an antibiotic agent, the method comprising the steps of:
(a) contacting a specimen obtained from the sample with an oligonucleotide probe or pair of probes in the absence of the agent and in the presence of amdinocillin, wherein the probe or pair of probes specifically hybridizes to a target sequence over the full length of the target sequence, wherein the target sequence comprises 25-35 contiguous nucleotides of mature ribosomal RNA (mrRNA) or ribosomal RNA spanning a splice site between a pre-ribosomal RNA (prRNA) tail and mrRNA;
(b) contacting a specimen obtained from the sample with the probe or pair of probes in the presence of the antibiotic agent and in the presence of amdinocillin;
(c) detecting the relative amounts of probe hybridization to the target sequence in the specimens of (a) and (b);
(d) identifying the sample as susceptible to antibiotic treatment if the amount of probe hybridization to the target sequence in step (b) is reduced relative to the amount of probe hybridization to the target sequence in step (a).

2. The method of claim 1, further comprising inoculating the specimen into a growth medium prior to the contacting of steps (a) and (b).

3. The method of claim 1, wherein the rRNA is 23 S rRNA.

4. The method of claim 3, wherein the target sequence is selected from:
(a) E. coli (all enterobacteriaceae) target sequence:
(SEQ ID NO: 1)
AATGAACCGTGAGGCTT|AACCTTACAACGCCGAAGCTGTTTTGGCGG
ATTG;
(b) Pseudomonas aeruginosa target sequence:
(SEQ ID NO: 2)
AATTGCCCGTGAGGCTT|GACCATATAACACCCAAACAATCTGACGATT
GT;
(c) Streptococcus pyogenes target sequence:
(SEQ ID NO: 3)
AATAGCTCGAGGACTT|ATCCAAAAAGAAATATTGACAACGTTACGGAT
TCTTG;
(d) Staphylococcus aureus target sequence:
(SEQ ID NO: 4)
AATCGATCGAAGACTT|AATCAAAATAAATGTTTTGCGAAGCAAAATC
ACTT;
wherein | indicates the splice site between prRNA and mRNA.

5. The method of claim 3, wherein the probe pair is selected from:
(a) E. coli (all enterobacteriaceae) probes:
(SEQ ID NO: 5)
5′-AAGCCTCACGGTTCATT
and
(SEQ ID NO: 6)
GGCGTTGTAAGGTT;
(b) Pseudomonas aeruginosa probes:
(SEQ ID NO: 7)
5′-AAGCCTCACGGGCAATT
and
(SEQ ID NO: 8)
GGTGTTATATGGTC;
(c) Streptococcus pyogenes probes:
(SEQ ID NO: 9)
AAGTCCTCGAGCTATT
and
(SEQ ID NO: 10)
ATTTCTTTTTGGAT;
and
(d) Staphylococcus aureus probes
(SEQ ID NO: 11)
AAGTCTTCGATCGATT
and
(SEQ ID NO: 12)
CATTTATTTTGATT.

6. The method of claim 1, wherein no pre-treatment of the specimen to deplete prRNA is performed prior to the contacting of steps (a) or (b).

7. The method of claim 1, wherein the detecting comprises an optical, electrochemical or immunological assay.

8. The method of claim 7, wherein the detecting comprises an electrochemical assay.

9. The method of claim 1, further comprising lysing the bacteria under conditions that release rRNA from the bacteria prior to the contacting of steps (a) and (b).

10. The method of claim 1, wherein the oligonucleotide probe or probes are each between about 10 to 50 nucleotides in length.

11. The method of claim 1, wherein the oligonucleotide probe is labelled with a detectable marker.

12. The method of claim 11, wherein the marker is selected from the group consisting of fluorescent label, a radioactive label, a luminescent label, an enzyme, biotin, thiol or a dye.

13. The method of claim 1, wherein the antibiotic agent is Rifampicin, Chloramphenicol, aminoglycosides, quinolones, or beta-lactam antibiotics.

14. A method for determining the antibiotic efficacy of a candidate antibiotic agent, the method comprising the steps of:
(a) contacting a specimen obtained from the sample with an oligonucleotide probe or pair of probes in the absence of the agent and in the presence of amdinocillin, wherein the probe or pair of probes specifically hybridizes to a target sequence over the full length of the target sequence, wherein the target sequence comprises 25-35 contiguous nucleotides of mature ribosomal RNA (mrRNA) or ribosomal RNA spanning a splice site between pre-ribosomal RNA (prRNA) tail and mrRNA;
(b) contacting a specimen obtained from the sample with the probe or pair of probes in the presence of the agent and in the presence of amdinocillin;
(c) detecting the relative amounts of probe hybridization to the target sequence in the specimens of (a) and (b);
(d) identifying the agent as effective if the amount of probe hybridization to the target sequence in step (b) is reduced by at least 10% relative to the amount of probe hybridization to the target sequence in step (a).

15. A method for monitoring the growth rate of a bacterial culture, the method comprising:
(a) contacting a specimen obtained from the culture with a probe or pair of probes that specifically hybridizes to a target sequence over the full length of the target sequence and in the presence of amdinocillin, wherein the target sequence comprises 25-35 contiguous nucleotides of bacterial ribosomal RNA (rRNA) spanning a splice site between pre-ribosomal RNA (prRNA) tail and mature ribosomal RNA (mrRNA);
(b) detecting the amount of probe hybridization to the target sequence in the specimen of (a) relative to an earlier time point or other control.
(c) identifying the culture as growing if the amount of probe hybridization to the target sequence in step (b) is increasing relative to the amount of probe hybridization to the target sequence at the earlier time point.

16. The method of claim 1, wherein the specimen comprises blood or serum obtained from a patient having or suspected of having a bacterial infection.

17. A method of determining whether a patient suffering from a bacterial infection has an infection caused by antibiotic-susceptible bacteria, the method comprising:
(a) administering amdinocillin and a beta-lactam antibiotic to the patient;
(b) monitoring the level of bacterial infection in the patient; and
(c) determining that the patient suffers from an infection caused by antibiotic-susceptible bacteria if the level of bacterial infection is reduced following the administering of (a).


SEQUENCE LISTING (50 sequences)

SEQ ID NO: 1 - DNA, 51 bp, Escherichia coli
aatgaaccgtgaggcttaaccttacaacgccgaagctgttttggcggattg

SEQ ID NO: 2 - DNA, 50 bp, Pseudomonas aeruginosa
aattgcccgtgaggcttgaccatataacacccaaacaatctgacgattgt

SEQ ID NO: 3 - DNA, 53 bp, Streptococcus pyogenes
aatagctcgaggacttatccaaaaagaaatattgacaacgttacggattcttg

SEQ ID NO: 4 - DNA, 51 bp, Staphylococcus aureus
aatcgatcgaagacttaatcaaaataaatgttttgcgaagcaaaatcactt

SEQ ID NO: 5 - DNA, 17 bp, Artificial Sequence
  Synthetic Probe
aagcctcacggttcatt

SEQ ID NO: 6 - DNA, 14 bp, Artificial Sequence
  Synthetic Probe
ggcgttgtaaggtt

SEQ ID NO: 7 - DNA, 17 bp, Artificial Sequence
  Synthetic Probe
aagcctcacgggcaatt

SEQ ID NO: 8 - DNA, 14 bp, Artificial Sequence
  Synthetic Probe
ggtgttatatggtc

SEQ ID NO: 9 - DNA, 16 bp, Artificial Sequence
  Synthetic Probe
aagtcctcgagctatt

SEQ ID NO: 10 - DNA, 14 bp, Artificial Sequence
  Synthetic Probe
atttctttttggat

SEQ ID NO: 11 - DNA, 16 bp, Artificial Sequence
  Synthetic Probe
aagtcttcgatcgatt

SEQ ID NO: 12 - DNA, 14 bp, Artificial Sequence
  Synthetic Probe
catttattttgatt

SEQ ID NO: 13 - DNA, 15 bp, Artificial Sequence
  Synthetic Probe
tttttcgtcttgcga

SEQ ID NO: 14 - DNA, 15 bp, Artificial Sequence
  Synthetic Probe
gagacttggtattca

SEQ ID NO: 15 - DNA, 15 bp, Artificial Sequence
  Synthetic Probe
gagacttggtattca

SEQ ID NO: 16 - DNA, 15 bp, Artificial Sequence
  Synthetic Probe
tttttcgtcttgcga

SEQ ID NO: 17 - DNA, 17 bp, Artificial Sequence
  Synthetic Probe
ttgagacttggtattca

SEQ ID NO: 18 - DNA, 17 bp, Artificial Sequence
  Synthetic Probe
tttttcgtcttgcgacg

SEQ ID NO: 19 - DNA, 19 bp, Artificial Sequence
  Synthetic Probe
tcttgagacttggtattca

SEQ ID NO: 20 - DNA, 19 bp, Artificial Sequence
  Synthetic Probe
tttttcgtcttgcgacgtt

SEQ ID NO: 21 - DNA, 21 bp, Artificial Sequence
  Synthetic Probe
actcttgagacttggtattca

SEQ ID NO: 22 - DNA, 21 bp, Artificial Sequence
  Synthetic Probe
tttttcgtcttgcgacgttaa

SEQ ID NO: 23 - DNA, 17 bp, Artificial Sequence
  Synthetic Probe
agacttggtattcattt

SEQ ID NO: 24 - DNA, 17 bp, Artificial Sequence
  Synthetic Probe
ttcgtcttgcgacgtta

SEQ ID NO: 25 - DNA, 19 bp, Artificial Sequence
  Synthetic Probe
tgagacttggtattcattt

SEQ ID NO: 26 - DNA, 19 bp, Artificial Sequence
  Synthetic Probe
ttcgtcttgcgacgttaag

SEQ ID NO: 27 - DNA, 21 bp, Artificial Sequence
  Synthetic Probe
cttgagacttggtattcattt

SEQ ID NO: 28 - DNA, 21 bp, Artificial Sequence
  Synthetic Probe
ttcgtcttgcgacgttaagaa

SEQ ID NO: 29 - DNA, 17 bp, Artificial Sequence
  Synthetic Probe
ctcttgagacttggtat

SEQ ID NO: 30 - DNA, 18 bp, Artificial Sequence
  Synthetic Probe
tcatttttcgtcttgcgb

SEQ ID NO: 31 - DNA, 19 bp, Artificial Sequence
  Synthetic Probe
cactcttgagacttggtat

SEQ ID NO: 32 - DNA, 19 bp, Artificial Sequence
  Synthetic Probe
tcatttttcgtcttgcgac

SEQ ID NO: 33 - DNA, 21 bp, Artificial Sequence
  Synthetic Probe
ttcactcttgagacttggtat

SEQ ID NO: 34 - DNA, 21 bp, Artificial Sequence
  Synthetic Probe
tcatttttcgtcttgcgacgt

SEQ ID NO: 35 - DNA, 19 bp, Artificial Sequence
  Synthetic Probe
tttgatgctcaaagaatta

SEQ ID NO: 36 - DNA, 21 bp, Artificial Sequence
  Synthetic Probe
tcaaactcttcaatttaaaag

SEQ ID NO: 37 - DNA, 21 bp, Artificial Sequence
  Synthetic Probe
tcaaactcttcaatttaaaag

SEQ ID NO: 38 - DNA, 19 bp, Artificial Sequence
  Synthetic Probe
tttgatgctcaaagaatta

SEQ ID NO: 39 - DNA, 17 bp, Artificial Sequence
  Synthetic Probe
gaggtgatccaaccgca

SEQ ID NO: 40 - DNA, 20 bp, Artificial Sequence
  Synthetic Probe
gaacgcttctttaaggtaag

SEQ ID NO: 41 - DNA, 20 bp, Artificial Sequence
  Synthetic Probe
gaacgcttctttaaggtaag

SEQ ID NO: 42 - DNA, 17 bp, Artificial Sequence
  Synthetic Probe
gaggtgatccaaccgca

SEQ ID NO: 43 - DNA, 14 bp, Artificial Sequence
  Synthetic Probe
ggcgttgtaaggtt

SEQ ID NO: 44 - DNA, 17 bp, Artificial Sequence
  Synthetic Probe
aagcctcacggttcatt

SEQ ID NO: 45 - DNA, 18 bp, Artificial Sequence
  Synthetic Probe
gttacgacttcaccccag

SEQ ID NO: 46 - DNA, 19 bp, Artificial Sequence
  Synthetic Probe
gttcccctacggttacctt

SEQ ID NO: 47 - DNA, 31 bp, Artificial Sequence
  Synthetic Probe
aatgaaccgtgaggcttaaccttacaacgcc

SEQ ID NO: 48 - DNA, 37 bp, Artificial Sequence
  synthetic probe
ctggggtgaagtcgtaacaaggtaaccgtaggggaac

SEQ ID NO: 49 - RNA, 155 nt, Escherichia coli
aguuugagaaguuaauuuucaaacugcgaguuucuuaauuugaagcauuacuuaaugcacaagugagaacucugaaccauaaguaaaaagcagaacgcugcaauucuuaggcauagaagcucacgcgugauguuucaugcgaagaaauuccauuc

SEQ ID NO: 50 - RNA, 64 nt, Escherichia coli
cgaaucagcgaauuggagugggcuucuacaaauuugucgaagccgcauuccaauucggagugcc